(19) United States
(12) Patent Application Publication
(10) Pub. No.: US 2025/0298019 A1
(43) Pub. Date: Sep. 25, 2025

(54) BORRELIA BURGDORFERI PEPTIDOGLYCAN DETECTION METHODS

(71) Applicant: VIRGINIA TECH INTELLECTUAL PROPERTIES, INC., Blacksburg, VA (US)

(72) Inventor: Brandon Jutras, Blacksburg, VA (US)

(21) Appl. No.: 18/709,394

(22) PCT Filed: Nov. 10, 2022
(86) PCT No.: PCT/US2022/079660

§ 371 Date: May 10, 2024

Related U.S. Application Data
(60) Provisional application No. 63/278,874, filed on Nov. 12, 2021.

(51) Int. Cl. G01N 33/569; A61K 45/00; C07K 16/12

(52) U.S. Cl. CPC G01N 33/56911; A61K 45/00; C07K 16/1207; G01N 2333/20; G01N 2400/02; G01N 2800/52; G01N 2800/56

(57) ABSTRACT

Described herein are assays, techniques, methods, and compositions capable of detecting a Borrelia burgdorferi microorganism, the causative agent of Lyme's disease.

### CROSS-REFERENCE TO RELATED APPLICATIONS

This application is the U.S. National Stage Application under 35 U.S.C. § 371 of Patent Cooperation Treaty Application No.: PCT/US2022/079660, filed on Nov. 10, 2022, which claims the benefit of and priority to U.S. Provisional Patent Application No. 63/278,874, filed on Nov. 12, 2021, the contents of which are incorporated by reference herein in their entireties.

### STATEMENT REGARDING FEDERALLY SPONSORED RESEARCH

This invention was made with government support under Grant No. R21AI159800 awarded by National Institutes of Health. The government has certain rights in the invention.

### SEQUENCE LISTING

This application contains a sequence listing filed in electronic form as an xml file entitled VTIP-0355WP_ST26.xml, created on Nov. 4, 2024 and having a size of 8,171 bytes. The content of the sequence listing is incorporated herein in its entirety.

### TECHNICAL FIELD

The subject matter disclosed herein is generally directed to assays and techniques for detecting infection with Borrelia burgdorferi (B. burgdorferi) organisms.

### BACKGROUND

The pathogenic spirochaete B. burgdorferi is estimated to cause more than 450,000 cases of Lyme disease each year in the U.S. alone. B. burgdorferi causes a bi-phasic infection with the acute stage being characterized by ‘flu-like’ symptoms, which is followed by a severe late-stage infection that can involve multiple organ systems and cause severe morbidity and can be fatal. Very little is known about what causes the clinical symptoms and there are few clinically relevant assays to test for infection, particularly active infection. As such there is a critical need for improved disease characterization and understanding and for methods of detecting B. burgdorferi for assessment and management of the infection and resulting Lyme's disease.

Citation or identification of any document in this application is not an admission that such a document is available as prior art to the present invention.

### SUMMARY

Described in certain example embodiments herein are methods of detecting a Borrelia burgdorferi (B. burgdorferi) organism, particularly an infection thereof, the method comprising: detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide.

In certain example embodiments, detecting comprises mass spectrometry, chromatography (e.g., gas, ion exchange, size-exclusion, liquid, high-performance, ultra-high performance, and/or the like), a PCR assay, an immunoassay or immunoseparation technique, electrophoresis, a periodate reaction, PAGE (native and denaturing), a size or mass separation technique, a charge separation technique, or any combination thereof, a resonance spectroscopy method (e.g., nuclear magnetic resonance), Raman spectroscopy, or any combination thereof, optionally wherein detecting comprises a PCR-immunoassay.

In some embodiments, detecting comprises contacting the sample with an antibody or fragment thereof capable of specifically binding the B. burgdorferi-specific peptidoglycan or fragment thereof. In some embodiments, the antibody or fragment thereof comprises or consists of a polypeptide having a sequence according to SEQ ID NO: 1, SEQ ID NO: 2, or both.

In certain example embodiments, the sample is a biological fluid sample, wherein the biological fluid sample is optionally, blood, plasma, serum, saliva, synovial fluid, cerebrospinal fluid, urine, lymph, sweat, stool, mucus, tears, or any combination thereof.

In certain example embodiments, the sample is from a subject having, has had, or is suspected of having Lyme's disease and/or B. burgdorferi infection.

In certain example embodiments, the method is effective of detecting B. burgdorferi during any stage of B. burgdorferi infection.

In certain example embodiments, the method further comprises diagnosing, monitoring, staging, and/or prognosing a B. burgdorferi infection and/or Lyme's disease in a subject from which the sample was obtained.

In certain example embodiments, the method does not detect other Borrelia species, other spirochetes, and/or other bacteria, and/or other microorganisms.

In certain example embodiments, the method further comprises treating a subject from which the sample was obtained for staging Lyme's disease and/or infection with a Borrelia burgdorferi (B. burgdorferi) organism or a symptom thereof, by administering to the subject an anti-infective agent, an anti-inflammatory agent, an analgesic, an antibody or fragment thereof, or any combination thereof, or any combination thereof. In some embodiments, the anti-infective agent comprises or consists of doxycycline, amoxicillin, cefuroxime, cefotaxime, azlocillin, penicillin, erythromycin, ceftriaxone, or any combination thereof. In some embodiments, treating comprises administering an antibody or fragment thereof capable of specifically binding the B. burgdorferi-specific peptidoglycan or fragment thereof or a pharmaceutical formulation thereof to the subject. In some embodiments, the antibody or fragment thereof comprises or consists of a polypeptide having a sequence according to SEQ ID NO: 1, SEQ ID NO: 2, or both.

In some embodiments, the method further comprises staging Lyme's disease, infection with a B. burgdorferi organism, or both, or a symptom thereof.

Described in certain example embodiments herein are methods of treating, diagnosing, prognosing, and/or staging Lyme's disease and/or infection with a Borrelia burgdorferi (B. burgdorferi) organism or a symptom thereof in a subject, the method comprising detecting, in a sample obtained from a subject that has had, has, or is suspected of having Lyme's disease and/or infection with B. burgdorferi, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; and administering an anti-infective agent, an anti-inflammatory agent, an analgesic, an antibody or fragment thereof, or any combination thereof to the subject. In some embodiments, the anti-infective agent comprises or consists of doxycycline, amoxicillin, cefuroxime, cefotaxime, azlocillin, penicillin, erythromycin, ceftriaxone, or any combination thereof. In some embodiments, the antibody or fragment thereof comprises or consists of one or more polypeptides each independently having a sequence according to SEQ ID NO: 1 or SEQ ID NO: 2.

In certain example embodiments, administering the treatment to a subject is oral, intermuscular, intravenous, intracerebroventricular, lumbar puncture, intra-articular, intraarterial, intraperitoneal, and/or any other suitable route of administration.

Described in certain example embodiments herein are kits comprising (a) one or more reagents capable of or adapted for detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; (b) one or more compositions or pharmaceutical formulations capable of treating a B. burgdorferi infection or a symptom thereof, wherein the one or more compositions or pharmaceutical formulations comprise an anti-infective agent, an anti-inflammatory agent, an analgesic, an antibody or fragment thereof, or any combination thereof, or (c) both (a) and (b).

In certain example embodiments, the antibody or fragment thereof comprises or consists of one or more polypeptides each independently having a sequence according to SEQ ID NO: 1 or SEQ ID NO: 2.

In certain example embodiments, the one or more reagents capable of or adapted for detecting comprises an antibody or fragment thereof, wherein the antibody or fragment thereof comprises or consists of one or more polypeptides each independently having a sequence according to SEQ ID NO: 1 or SEQ ID NO: 2.

In certain example embodiments, the anti-infective agent comprises or consists of doxycycline, amoxicillin, cefuroxime, cefotaxime, azlocillin, penicillin, erythromycin, ceftriaxone, or any combination thereof.

Described in certain example embodiments herein are antibodies or fragments thereof comprising one or more polypeptides each independently having a sequence according to SEQ ID NO: 1 or SEQ ID NO: 2. In certain example embodiments, the antibody is capable of specifically binding a B. burgdorferi-specific peptidoglycan or fragment thereof.

In certain example embodiments, the antibody is capable of specifically binding B. burgdorferi-specific peptidoglycan or fragment comprising a GlcNAc-GlcNAc-MurNAc (GGM) trisaccharide.

Described in certain example embodiments herein are pharmaceutical formulation comprising an antibody or fragment thereof of the present disclosure; and a pharmaceutically acceptable carrier. In some embodiments, the antibodies or fragments thereof comprise one or more polypeptides each independently having a sequence according to SEQ ID NO: 1 or SEQ ID NO: 2.

Described in certain example embodiments herein are methods of treating a B. Burgdorefi infection or a symptom thereof in a subject in need thereof, the method comprising administering the antibody of the present disclosure or a pharmaceutical formulation thereof to the subject in need thereof. In some embodiments, the antibodies or fragments thereof comprise one or more polypeptides each independently having a sequence according to SEQ ID NO: 1 or SEQ ID NO: 2.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—Elucidating the peptidoglycan glycan strand composition of B. burgdorferi. FIG. 1A, MS/MS of the GlcNAc-MurNAc-AlaGluOrnGly muropeptide from B. burgdorferi 5A11 cultured in unlabelled (grey) and [1-¹³C]ManNAc (blue), respectively. Fragmentation data confirm the location of the labelled carbon resides in the glycan component and not the stem peptide. FIG. 1B, Monosaccharide analysis of purified peptidoglycan isolated from B. burgdorferi 5A11 and E. coli K-12. results were compared with reference standards GlcNAc, MurNAc, ManNAc and GalNAc (below). The inset table highlights the molar percentage of MurNAc present in each bacterial sample. FIG. 1C, LC-MS chromatogram of B. burgdorferi 5A11 peptidoglycan. B. burgdorferi peptidoglycan was purified, digested with mutanolysin and analysed by LC-MS. Each peak corresponds to one or more muropeptides of interest; peaks are labelled as red (GlcNAc-MurNAc muropeptides) or blue (HexNAc-GlcNAc-MurNAc muropeptides). Co-eluting peaks can be found in FIG. 13.

FIG. 2A-2D—B. burgdorferi peptidoglycan glycan strands contain the trisaccharide G-G-anhM. FIG. 2A, LC-MS chromatogram of unlabelled B. burgdorferi 5A1 1 peptidoglycan. Total ion chromatogram is shown in black with an unlabelled and [1-¹³C]G-G-anhM muropeptide overlaid in pink and blue, respectively. FIG. 2B, LC-MS chromatogram of [1-¹³C]GlcNAc metabolically labelled B. burgdorferi 5A11 peptidoglycan producing a mass shift corresponding to a G-G-anhM muropeptide. B. burgdorferi was cultured with unlabelled GlcNAc or [1-¹³C]GlcNAc before peptidoglycan purification and LC-MS analysis. The proposed G-G-anhM species 1,053 m/z in unlabelled peptidoglycan and the shifted mass to the predicted 1,056 m/z when labelled with [1-¹³C]GlcNAc are shown. FIG. 2C, The ¹³C-labelled NMr of the anomeric region of 1-¹³C-labelled B. burgdorferi peptidoglycan and an N,N′,N″-triacetylchitotriitol reference standard with the highlighted region (light blue) indicating a putative chemical shift for the non-reducing-end anomeric carbon. FIG. 2D, A comparative muropeptide analysis of peptidoglycan isolated from three clonal derivatives of B. burgdorferi and one strain of B. hermsii. Three laboratory strains of B. burgdorferi, two fully infectious clones of the B31-type strain (5A11, green; 5A3, purple) and non-infectious (n.i.) derivative of 5A3, as well as B. hermsii were cultured to mid-log, peptidoglycan was purified, digested and muropeptide profiles compared by LC. All samples contained similar levels of G-G-anhM muropeptides (*).

FIG. 3A-3B—Comparative analysis of muropeptide profiles obtained from B. burgdorferi A3 and chitobiose transport mutant A3/chbC. FIG. 3A, Principal component (PC) analysis of 37 distinct muropeptide features collected from LC-MS data of three biological replicates, from WT A3 strain (tan) and A3/chbC (teal) peptidoglycan. FIG. 3B, representative LC spectra from our comparative muropeptide analysis (in a) in which the amount of purified and injected peptidoglycan was normalized by the total number of cells present in each culture.

FIG. 4A-4C—Impact of chitobiose (GlcNAc-GlcNAc) transport on peptidoglycan and cell-wall stress. FIG. 4A, Comparative AFM analysis of purified peptidoglycan. Peptidoglycan from both the WT A3 strain and the A3/chbC chitobiose mutant transporter strain was deposited on mica and topological features imaged in AM-FM mode. Height features for each image are shown as colour maps (right, as represented in greyscale) in nanometres. Scale bars, 5 μm (upper panel), 500 nm (middle panel) and 50 nm (lower panel). FIG. 4B-4C, recovery after cell-wall stress. WT A3 and A3/chbC strains were exposed to 0.25 M NaCl (544 mosmol) (FIG. 4B) or 1 mg ml-1 of lysozyme (FIG. 4C) for 24 h. After removing exogenous stress, cultures were plated in quadruplicate and colony-forming units were determined 9 d later. Statistical significance (*) was determined by two-tailed, unpaired Student's t-test (NaCl: P=1.37×10-5; lysozyme: P=0.009).

FIG. 5A-5C—Morphological and motility defects in A3/chbC mutant bacteria. FIG. 5A, Comparative, quantitative, population-level morphological analysis of A3 and A3/chbC strains. Both strains were cultured to mid-log(exponential growth), fixed with paraformaldehyde to preserve cellular helicity and imaged on agarose pads by phase-contrast microscopy. Scale bar, 5 μm. FIG. 5B, Morphometric, population-level analysis of differences in helical pitch between strains estimated by the object analysis feature roundness. Box plots from values attained from n=360 (A3) and n=481 (A3/chbC) strains are shown. Each dot indicates the values attained from an individual cell. Statistical significance was determined using the two-tailed, unpaired Student's t-test (P=6.67×10-9). FIG. 5C, Swarm plate assay to measure differences in bacterial motility. Liquid A3 and A3/chbC cultures were enumerated and equal amounts used to inoculate the same semisoft agar plate, equidistant from each other. After 5 d, swarming distance was measured from five replicate plates. Statistical significance (*) was determined using the two-tailed, unpaired Student's t-test (P=0.0031).

FIG. 6A-6D—Biophysical properties of B. burgdorferi peptidoglycan with reduced levels of G-G-anhM. FIG. 6A, AM-FM topological mapping (upper) and elasticity measurements (lower) using the Hertz contact model on purified peptidoglycan sacculi from each strain. Note that measurements collected for each sample had dramatically different force ranges, which is reflected in colour maps as represented in greyscale (below). These images represent data collected from eight independent sacculi per sample. Scale bar, 400 nm. FIG. 6B, Line-scan analysis of force measurements collected from each pixel in seven independent sacculi per sample. Statistical significance (*) was assessed using the two-tailed, unpaired Student's t-test (P=5.3×10-5). FIG. 6C, The same line scans in b were used to measure pixel-level height differences in each sample. Statistical significance (*) was assessed using the two-tailed, unpaired Student's t-test (P=0.0028). FIG. 6D, Fold-change of the elasticity of A3/chbC peptidoglycan, relative to A3, normalized by peptidoglycan height.

FIG. 7—Growth of 5A11 B. burgdorferi in BSK-II complete media supplemented with GlcNAc, ManNAc, or GalNAc. Growth of 5A11 B. burgdorferi in BSK-II complete media supplemented with GlcNAc, ManNAc, or GalNAc. Values represent the mean and standard deviation of three independent cultures.

FIG. 8A-8B—LCMS chromatogram of borohydride reduced 5A11 B. burgdorferi peptidoglycan. FIG. 8A) LCMS chromatogram of borohydride reduced 5A11 B. burgdorferi peptidoglycan (left) and corresponding MS/MS spectra of borohydride reduced muropeptide 5 (right). The TIC is shown in black and the abundance of borohydride reduced muropeptide 5 is shown in orange (1053.4277-1053.4699 m/z scanned). The precursor ion selected for MS/MS is shown in orange. FIG. 8B) LCMS chromatogram of borodeuteride reduced 5A11 B. burgdorferi peptidoglycan (left) and corresponding MS/MS spectra of muropeptide 5 (right). The TIC is shown in black and the abundance of borodeuteride reduced muropeptide 5 is shown in purple (1053.4289-1053.4711 m/z scanned). The precursor ion selected for MS/MS is shown in purple.

FIG. 9A-9B—LC-MS Analysis of a Chitobiose Standard. LC-MS Analysis of a Chitobiose Standard. FIG. 9A, LC-MS traces using Selected Ion Monitoring (SIM) of the sodiated ion, and the Total Ion Chromatograms (TIC) for MS2 analysis of the protonated and sodiated species. FIG. 9B, MS2 spectra for the protonated (top) and sodiated (bottom) forms of chitobiose. Spectra summed over the time window indicated in the grey box (4.80-5.17 min).

FIG. 10A-10B—LC-MS Analysis of Commercial Autohydrolyzed Yeast (Yeastolate). FIG. 10A, LC-MS Analysis of Commercial Autohydrolyzed Yeast (Yeastolate). A) LC-MS traces using Selected Ion Monitoring (SIM) of the m/z values for chitobiose as the sodiated ion, and the Total Ion Chromatograms (TIC) for MS2 analysis of the protonated and sodiated species. FIG. 10B, MS2 spectra for the m/z values of the protonated (top) and sodiated (bottom) forms of chitobiose. Spectra summed over the time window indicated in the grey box (4.80-5.11 min).

FIG. 11—LCMS chromatograms by muropeptide—Total ion counts (TICs) are shown in black with the [M+H]⁺ and [M+Na]⁺ for each muropeptide shown in blue and purple, respectively. *Muropeptides containing G-G-anhM are denoted by an asterick.

FIG. 12—Analysis of muropeptides present in 5A11 B. burgdorferi peptidoglycan.

The figures herein are for illustrative purposes only and are not necessarily drawn to scale. γ—As previously identified in Jutras et. al., 2019, PNAS. NM denotes newly identified muropeptides. * Muropeptide contains G-G-anhM.

FIG. 13—Muropeptide-containing peaks from 5A11 B. burgdorferi peptidoglycan analyzed via LCMS. * Muropeptide contains G-G-anhM.

FIG. 14—Theoretical vs. observed m/z for each muropeptide after mass correction using an internal standard. * Muropeptide contains G-G-anhM.

FIG. 15A-15C—(FIG. 15A) MS1 spectra generated from scanning retention times of 1.542-1.908 minutes. (FIG. 15B) The structure of muropeptide 1. Cleavages with resulting m/z fragments are shown in red. (FIG. 15C) MS2 obtained from targeting precursor ion 667.3145 [M+H]⁺. Red fragments generated in FIG. 15B correspond to observed MS2 fragments in FIG. 15C.

FIG. 16A-16C—(FIG. 16A) MS1 spectra generated from scanning retention times of 2.788-3.162 minutes. (FIG. 16B) The structure of muropeptide 2a. Cleavages with resulting m/z fragments are shown in red. (FIG. 16C) MS2 obtained from targeting precursor ion 870.3902 [M+H]⁺. Red fragments generated in FIG. 16B correspond to observed MS2 fragments in FIG. 16C.

FIG. 17A-17C—(FIG. 17A) MS1 spectra generated from scanning retention times of 3.235-3.488 minutes. (FIG. 17B) The structure of muropeptide 2b. Cleavages with resulting m/z fragments are shown in red. (FIG. 17C) MS2 obtained from targeting precursor ion 870.3902 [M+H]⁺. Red fragments generated in FIG. 17B correspond to observed MS2 fragments in FIG. 17C.

FIG. 18A-18C—(FIG. 18A) MS1 spectra generated from scanning retention times of 6.495-6.735 minutes. (FIG. 18B) The structure of muropeptide 3. Cleavages with resulting m/z fragments are shown in red. (FIG. 18C) MS2 obtained from targeting precursor ion 555.2676 [M+2H]⁺². Red fragments generated in FIG. 18B correspond to observed MS2 fragments in FIG. 18C.

FIG. 19A-19C—(FIG. 19A) MS1 spectra generated from scanning retention times of 7.575-8.042 minutes. (FIG. 19B) The structure of muropeptide 4. Cleavages with resulting m/z fragments are shown in red. (FIG. 19C) MS2 obtained from targeting precursor ion 656.8094 [M+2H]⁺². Red fragments generated in B correspond to observed MS2 fragments in FIG. 19C.) MS1 spectra generated from scanning retention times of 7.575-8.042 minutes. (FIG. 19B) The structure of muropeptide 4. Cleavages with resulting m/z fragments are shown in red. (FIG. 19C) MS2 obtained from targeting precursor ion 656.8094 [M+2H]⁺². Red fragments generated in FIG. 19B correspond to observed MS2 fragments in FIG. 19C.

FIG. 20A-20C—(FIG. 20A) MS1 spectra generated from scanning retention times of 8.775-9.162 minutes. (FIG. 20B) The structure of muropeptide 5. Cleavages with resulting m/z fragments are shown in red. (FIG. 20C) MS2 obtained from targeting precursor ion 1053.4426 [M+H]⁺. Red fragments generated in FIG. 20B correspond to observed MS2 fragments in FIG. 20C.

FIG. 21A-21C—(FIG. 21A) MS1 spectra generated from scanning retention times of 8.775-9.162 minutes. (FIG. 21B) The structure of muropeptide 6a. Cleavages with resulting m/z fragments are shown in red. (FIG. 21C) MS2 obtained from targeting precursor ion 850.3676 [M+H]⁺. Red fragments generated in FIG. 21B correspond to observed MS2 fragments in FIG. 21C.

FIG. 22A-22C—(FIG. 22A) MS1 spectra generated from scanning retention times of 11.352-11.577 minutes. (FIG. 22B) The structure of muropeptide 6b. Cleavages with resulting m/z fragments are shown in red. (FIG. 22C) MS2 obtained from targeting precursor ion 850.3676 [M+H]⁺. Red fragments generated in FIG. 22B correspond to observed MS2 fragments in FIG. 22C.

FIG. 23A-23C—(FIG. 23A) MS1 spectra generated from scanning retention times of 11.527-11.727 minutes. (FIG. 23B) The structure of muropeptide 7. Cleavages with resulting m/z fragments are shown in red. (FIG. 23C) MS2 obtained from targeting precursor ion 748.3359 [M+2H]⁺². Red fragments generated in FIG. 23B correspond to observed MS2 fragments in FIG. 23C.

FIG. 24A-24C—(FIG. 24A) MS1 spectra generated from scanning retention times of 11.827-12.027 minutes. (FIG. 24B) The structure of muropeptide 8a. Cleavages with resulting m/z fragments are shown in red. (FIG. 24C) MS2 obtained from targeting precursor ion 646.7963 [M+2H]⁺². Red fragments generated in FIG. 24B correspond to observed MS2 fragments in FIG. 24C.

FIG. 25A-25C—(FIG. 25A) MS1 spectra generated from scanning retention times of 11.827-12.027 minutes. (FIG. 25B) The structure of muropeptide 9a. Cleavages with resulting m/z fragments are shown in red. (FIG. 25C) MS2 obtained from targeting precursor ion 693.8277 [M+2H]⁺². Red fragments generated in FIG. 25B correspond to observed MS2 fragments in FIG. 25C.

FIG. 26A-26C—(FIG. 26A) MS1 spectra generated from scanning retention times of 12.552-12.752 minutes. (FIG. 26B) The structure of muropeptide 9b. Cleavages with resulting m/z fragments are shown in red. (FIG. 26C) MS2 obtained from targeting precursor ion 693.8277 [M+2H]⁺². Red fragments generated in FIG. 26B correspond to observed MS2 fragments in FIG. 26C.

FIG. 27A-27C—(FIG. 27A) MS1 spectra generated from scanning retention times of 12.552-12.752 minutes. (FIG. 27B) The structure of muropeptide 10a. Cleavages with resulting m/z fragments are shown in red. (FIG. 27C) MS2 obtained from targeting precursor ion 795.3674 [M+2H]⁺². Red fragments generated in FIG. 27B correspond to observed MS2 fragments in FIG. 27C.

FIG. 28A-28C—(FIG. 28A) MS1 spectra generated from scanning retention times of 13.127-13.427 minutes. (FIG. 28B) The structure of muropeptide 10b. Cleavages with resulting m/z fragments are shown in red. (FIG. 28C) MS2 obtained from targeting precursor ion 795.3674 [M+2H]⁺². Red fragments generated in FIG. 28B correspond to observed MS2 fragments in FIG. 28C.

FIG. 29A-29C—(FIG. 29A) MS1 spectra generated from scanning retention times of 13.127-13.427 minutes. (FIG. 29B) The structure of muropeptide 11. Cleavages with resulting m/z fragments are shown in red. (FIG. 29C) MS2 obtained from targeting precursor ion 896.9035 [M+H]⁺². Red fragments generated in FIG. 29B correspond to observed MS2 fragments in FIG. 29C.

FIG. 30A-30C—(FIG. 30A) MS1 spectra generated from scanning retention times of 13.667-13.927 minutes. (FIG. 30B) The structure of muropeptide 8b. Cleavages with resulting m/z fragments are shown in red. (FIG. 30C) MS2 obtained from targeting precursor ion 646.7963 [M+2H]⁺². Red fragments generated in FIG. 30B correspond to observed MS2 fragments in FIG. 30C.

FIG. 31A-31C—(FIG. 31A) MS1 spectra generated from scanning retention times of 15.262-15.512 minutes. (FIG. 31B) The structure of muropeptide 12a. Cleavages with resulting m/z fragments are shown in red. (FIG. 31C) MS2 obtained from targeting precursor ion 886.8902 [M+H]⁺². Red fragments generated in FIG. 31B correspond to observed MS2 fragments in FIG. 31C.

FIG. 32A-32C—(FIG. 32A) MS1 spectra generated from scanning retention times of 15.745-15.862 minutes. (FIG. 32B) The structure of muropeptide 13. Cleavages with resulting m/z fragments are shown in red. (FIG. 32C) MS2 obtained from targeting precursor ion 988.4299 [M+H]⁺². Red fragments generated in FIG. 32B correspond to observed MS2 fragments in FIG. 32C.

FIG. 33A-33C—(FIG. 33A) MS1 spectra generated from scanning retention times of 15.912-16.085 minutes. (FIG. 33B) The structure of muropeptide 14a. Cleavages with resulting m/z fragments are shown in red. (FIG. 33C) MS2 obtained from targeting precursor ion 785.3512 [M+H]⁺². Red fragments generated in FIG. 33B correspond to observed MS2 fragments in FIG. 33C.

FIG. 34A-34C—(FIG. 34A) MS1 spectra generated from scanning retention times of 16.342-16.470 minutes. (FIG. 34B) The structure of muropeptide 12b. Cleavages with resulting m/z fragments are shown in red. (FIG. 34C) MS2 obtained from targeting precursor ion 886.8902 [M+H]⁺². Red fragments generated in FIG. 34B correspond to observed MS2 fragments in FIG. 34C.

FIG. 35A-35C—(FIG. 35A) MS1 spectra generated from scanning retention times of 17.392-17.522 minutes. (FIG. 35B) The structure of muropeptide 14b. Cleavages with resulting m/z fragments are shown in red. (FIG. 35C) MS2 obtained from targeting precursor ion 785.3512 [M+H]⁺². Red fragments generated in FIG. 35B correspond to observed MS2 fragments in FIG. 35C.

FIG. 36A-36C—(FIG. 36A) MS1 spectra generated from scanning retention times of 17.717-17.890 minutes. (FIG. 36B) The structure of muropeptide 14c. Cleavages with resulting m/z fragments are shown in red. (FIG. 36C) MS2 obtained from targeting precursor ion 785.3512 [M+H]⁺². Red fragments generated in FIG. 36B correspond to observed MS2 fragments in FIG. 36C.

FIG. 37A-37C—(FIG. 37A) MS1 spectra generated from scanning retention times of 17.717-17.890 minutes. (FIG. 37B) The structure of muropeptide 12c. Cleavages with resulting m/z fragments are shown in red. (FIG. 37C) MS2 obtained from targeting precursor ion 886.8902 [M+H]⁺². Red fragments generated in FIG. 37B correspond to observed MS2 fragments in FIG. 37C.

FIG. 38A-38C—(FIG. 38A) MS1 spectra generated from scanning retention times of 18.370-18.570 minutes. (FIG. 38B) The structure of muropeptide 15a. Cleavages with resulting m/z fragments are shown in red. (FIG. 38C) MS2 obtained from targeting precursor ion 978.417 [M+H]⁺². Red fragments generated in FIG. 38B correspond to observed MS2 fragments in FIG. 38C.

FIG. 39A-39C—(FIG. 39A) MS1 spectra generated from scanning retention times of 18.917-19.130 minutes. (FIG. 39B) The structure of muropeptide 16a. (FIG. 39C) MS2 obtained from targeting precursor ion 1246.5609 [M+H]⁺².

FIG. 40A-40C—(FIG. 40A) MS1 spectra generated from scanning retention times of 19.197-19.330 minutes. (FIG. 40B) The structure of muropeptide 17a. Cleavages with resulting m/z fragments are shown in red. (FIG. 40C) MS2 obtained from targeting precursor ion 876.87755 [M+H]⁺². Red fragments generated in FIG. 40B correspond to observed MS2 fragments in FIG. 40C.

FIG. 41A-41C—(FIG. 41A) MS1 spectra generated from scanning retention times of 19.650-19.877 minutes. (FIG. 41B) The structure of muropeptide 15b. Cleavages with resulting m/z fragments are shown in red. (FIG. 41C) MS2 obtained from targeting precursor ion 978.4168 [M+H]⁺². Red fragments generated in FIG. 41B correspond to observed MS2 fragments in FIG. 41C.

FIG. 42A-42C—(FIG. 42A) MS1 spectra generated from scanning retention times of 20.215-20.590 minutes. (FIG. 42B) The structure of muropeptide 16b. (FIG. 42C) MS2 obtained from targeting precursor ion 1246.5609 [M+H]⁺².

FIG. 43A-43C—(FIG. 43A) MS1 spectra generated from scanning retention times of 20.457-20.590 minutes. (FIG. 43B) The structure of muropeptide 17b. Cleavages with resulting m/z fragments are shown in red. (FIG. 43C) MS2 obtained from targeting precursor ion 876.87755 [M+H]⁺². Red fragments generated in FIG. 43B correspond to observed MS2 fragments in FIG. 43C.

FIG. 44A-44C—(FIG. 44A) MS1 spectra generated from scanning retention times of 21.800-22.000 minutes. FIG. 44B) The structure of muropeptide 17c. Cleavages with resulting m/z fragments are shown in red. (FIG. 44C) MS2 obtained from targeting precursor ion 876.87755 [M+H]⁺². Red fragments generated in FIG. 44B correspond to observed MS2 fragments in FIG. 44C.

FIG. 45—A comparison of plasmid content in 5A11, 5A3, 5A3 N.I., and 5A3/chbC B. burgdorferi strains.

FIG. 46—In addition to the clean deletion of chbC, 5A3/chbC had the following single nucleotide polymorphisms, relative to 5A3.

FIG. 47A-47B—FIG. 47A) Features generated in RamCluster used for the comparison of G-G-anhM content in peptidoglycan from A3 and A3/chbC B. burgdorferi. Each feature is composed of a retention time and at least two peaks that correspond to an individual m/z. All features listed were used in the analysis. Features designated with an asterisk contain peaks corresponding to G-G-anhM. Features not listed (feature c07, c08, and c32-c35) were manually omitted—they represented adducts and redundancies that were not filtered out by RamClustR. A further breakdown of the peaks that compose each feature can be found in Supplemental Dataset 1. FIG. 47B) Relative comparison of G-G-anhM intensity in 5A3 and 5A3/chbC B. burgdorferi. All features were divided into two groups: G-G-anhM (features C03 & C11, designated by asterisk) or G-M (all other features).

FIG. 48—Thresholds applied to phase-contrast micrographs containing B. burgdorferi A3 (left) and A3/chbC (right). Thresholds were generated using Fiji. Scale bars are 5 μm.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, _{2nd}ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g., plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

The pathogenic spirochaete B. burgdorferi is estimated to cause more than 450,000 cases of Lyme disease each year in the U.S. alone. B. burgdorferi causes a bi-phasic infection with the acute stage being characterized by ‘flu-like’ symptoms, which is followed by a severe late-stage infection that can involve multiple organ systems and cause severe morbidity and can be fatal. Very little is known about what causes the clinical symptoms and there are few clinically relevant assays to test for infection, particularly active infection. As such there is a critical need for improved disease characterization and understanding and for methods of detecting B. burgdorferi for assessment and management of the infection and resulting Lyme's disease.

With that said, embodiments disclosed herein can provide methods of detecting a Borrelia burgdorferi (B. burgdorferi) organism, particularly an infection thereof, that can include detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Methods of Detecting B. burgdorferi and Uses Thereof

Described in certain embodiments herein are methods of detecting a Borrelia burgdorferi (B. burgdorferi) organism or an infection thereof. In some embodiments, the method includes detecting, in a sample, a B. burgdorferi-specific peptidoglycan or fragment thereof. In some embodiments, the B. burgdorferi-specific peptidoglycan or fragment thereof is composed all or in part of a GlcNAc-GlcNAc-MurNAc (GGM) trisaccharide.

The step of detecting can include any suitable method or techniques for detecting peptidoglycans or fragments thereof. In some embodiments, detecting includes mass spectrometry, chromatography, a polymerase chain reaction (PCR)-based assay, an immunoassay, immunoseparation, electrophoresis, a periodate reaction, size-based separation, a mass separation technique, a charge separation technique, resonance spectroscopy, Raman spectroscopy, or any combination thereof. In some embodiments, detecting includes performing a PCR-immunoassay (see e.g., Chang et al., Anal Chim Acta. 2016 Mar. 3; 910:12-24).

In some embodiments, the step of detecting includes contacting the sample with an antibody or fragment thereof capable of specifically binding the B. burgdorferi-specific peptidoglycan or fragment thereof. In some embodiments, the antibody or fragment thereof is composed of one or more polypeptide each independently having a sequence according to SEQ ID NO: 1 or SEQ ID NO: 2.

Clone 1A4-H (G-G-anhM mAb) Heavy chain
(SEQ ID NO: 1)
METGLRWLLLVAVLKGVQCQSLEESGGRLVTPGTPLTLTCTVSGFSLSS
YAVIWVRQAPGEGLEYIGIIFGGGKTYYASWAKGRFTISKTSTTVDLRI
TSPTTEDTATYFCVRDDWSRYLWGQGTLVTVSSGQPKAPSVFPLAPCCG
DTPSSTVTLGCLVKGYLPEPVTVTWNSGTLTNGVRTFPSVRQSSGLYSL
SSVVSVTSSSQPVTCNVAHPATNTKVDKTVAPSTCSKPMCPPPELPGGP
SVFIFPPKPKDTLMISRTPEVTCVVVDVSQDDPEVQFTWYINNEQVRTA
RPPLREQQFNSTIRVVSTLPIAHQDWLRGKEFKCKVHNKALPAPIEKTI
SKARGQPLEPKVYTMGPPREELSSRSVSLTCMINGFYPSDISVEWEKNG
KAEDNYKTTPTVLDSDGSYFLYSKLSVPTSEWQRGDVFTCSVMHEALHN
HYTQKSISRSPGK
Clone 1A4-H (G-G-anhM mAb) Light chain
(SEQ ID NO: 2)
MDTRAPTQLLGLLLLWLPGATFAQVLTQTASPVSAAVGSTVTINCQSSE
SVYSNNYLSWFHQKPGQPPKQLIYSASTLASGVPSRFKGSGSGTQFTLT
ISDVQCDDAATYYCLGNYGCSSADCRAFGGGTEVVVKGDPVAPTVLIFP
PAADQVATGTVTIVCVANKYFPDVTVTWEVDGTTQTTGIENSKTPQNSA
DCTYNLSSTLTLTSTQYNSHKEYTCKVTQGTTSVVQSFNRGDC

Detection of binding of the antibody or fragment thereof can be detected by any conventional detection technique or method, such as those routinely used in immunoassays. In some embodiments, the antibody or fragment thereof is labeled. Exemplary labels include, without limitation, radioisotopes and optically active labels (e.g., fluorescent labels, dyes, near-infrared labels, and/or the like, which are generally known in the art). In some embodiments, specific binding detecting is amplified by use of a secondary antibody that binds to the antibody or fragment thereof. Such techniques and compositions are also generally known in the art.

In some embodiments, the sample is a biological sample. In some embodiments, the biological sample is a biological fluid sample or a bodily fluid sample. In some embodiments, biological fluid or bodily fluid sample is whole blood, plasma, serum, saliva, synovial fluid, cerebrospinal fluid, urine, lymph, sweat, stool, mucus, tears, or any combination thereof.

In some embodiments, the sample is 1-1,000 pL, nL, μL, or mL. In some embodiments, the sample is 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 pL, nL, μL, or mL.

The subject can be any organism capable of being infected with or otherwise harboring (such as a vector) B. burgdoferi. In some embodiments, the subject is a human. In some embodiments, the subject is a non-human mammal. In some embodiments, the subject is a canine, feline, equine, ovine, porcine, bovine, or cervine. In some embodiments, the subject is a tick. In some embodiments, the sample is from a subject having, that has had, or is suspected of having Lyme's disease and/or a B. burgdorferi infection. In some embodiments, the subject is asymptomatic. In some embodiments, the subject is symptomatic.

In some embodiments, the method is effective in detecting B. burgdorferi during one or more stages of B. burgdorferi infection or Lyme's disease. n some embodiments, method is effect in detecting B. burgdorferi during any stage of B. burgdorferi infection. In some embodiments, the method is effective to detect B. burgdorferi during the early localized stage, the early disseminated stage, the late disseminated stage, or any combination thereof.

In some embodiments, the method further includes diagnosing, monitoring, staging, and/or prognosing a B. burgdorferi infection and/or Lyme's disease or a symptom thereof in a subject from which the sample was obtained. In some embodiments, the method includes obtaining two or more samples from the same subject, where one or more samples is/are obtained at two or more different time points. In some embodiments, any two points are 1-100 or more minutes, days, weeks, months, or years apart from each other. In some embodiments, the amount and/or types of peptidoglycan or fragment thereof detected in the sample is to determine a diagnosis, prognosis and used to monitor Lyme's disease or B. burgdorferi infection progression or response to a treatment or therapy.

In some embodiments, increased levels or amounts of B. burgdorferi peptidoglycan(s) or fragments thereof, such as a GGM) trisaccharide, compared to a suitable control indicates Lyme's disease and/or B. burgdorferi infection and/or lack of or a poor response to treatment. In some embodiments, increasing levels (such as those compared between two or more samples over time) of B. burgdorferi peptidoglycan(s) or fragments thereof, are indicative of disease progression and/or poor or a lack of response to treatment. In some embodiments, decreased or undetectable levels or amounts of B. burgdorferi peptidoglycan(s) or fragments thereof, such as a GGM) trisaccharide, compared to a suitable control indicates no Lyme's disease and/or B. burgdorferi infection, and/or good response to treatment or that treatment was effective. In some embodiments, decreasing levels or amounts (such as those compared between two or more samples over time) of B. burgdorferi peptidoglycan(s) or fragments thereof, are indicative of clearing of the B. burgdorferi organism and/or a good response to a treatment and/or an effective treatment. A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

In some embodiments, the method further includes staging Lyme's disease, infection with a Borrelia burgdorferi organism, or both, or a symptom thereof. The term “staging” is used in accordance with its commonplace use in the medical and/or veterinary fields, and refers to identifying what stage or phase of a disease progression a subject is in. In some embodiments, the level, amount, and/or type of B. burgdorferi peptidoclycan indicates alone or when considered with one or more other disease indicators the stage of Lyme's disease or B. burgdorferi infection as subject is in. In some embodiments, the subject is asymptomatic.

In some embodiments, the method is specific to detecting B. burgdorferi. In some embodiments, the method does not detect other Borrelia species, other spirochetes, and/or other bacteria, and/or other microorganisms.

In some embodiments, the method further includes treating a B. burgdorferi infection, Lyme's disease, or both or a symptom thereof in a subject from which the sample was obtained by administering to the subject an anti-infective agent, an anti-inflammatory agent, an analgesic, an antibody or fragment thereof, or any combination thereof.

In some embodiments, treating includes administering an antibody or fragment thereof capable of specifically binding the B. burgdorferi-specific peptidoglycan or fragment thereof or a pharmaceutical formulation thereof to the subject. In some embodiments, the antibody or fragment thereof is composed of or includes one or more a polypeptides each independently having a sequence according to SEQ ID NO: 1 or SEQ ID NO: 2.

Described in some embodiments, are methods of treating, diagnosing, prognosing, and/or staging Lyme's disease and/or infection with a Borrelia burgdorferi (B. burgdorferi) organism, and/or a symptom thereof, the method comprising: detecting, in a sample obtained from a subject that has had, has, or is suspected of having Lyme's disease and/or infection with B. burgdorferi, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; and administering an anti-infective agent, an anti-inflammatory agent, an analgesic, an antibody or fragment thereof, or any combination thereof to the subject.

Administration can be by any suitable route. In some embodiments, administering is oral, intermuscular, intravenous, intracerebroventricular, lumbar puncture, intra-articular, intraarterial, or intraperitoneal.

In some embodiments, the anti-infective agent comprises doxycycline, amoxicillin, cefuroxime, cefotaxime, azlocillin, penicillin, erythromycin, ceftriaxone, or any combination thereof. Other exemplary anti-infectives are described in greater detail herein.

Suitable antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable analgesics include, but are not limited to, paracetamol/acetaminophen, nonsteroidal anti-inflammatories (e.g., ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals. Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

B. burgdorferi Antibodies and Fragments Thereof Antibodies and Fragments thereof

Described in several exemplary antibodies herein are antibodies or fragments thereof comprising one or more polypeptides each independently having a sequence according to SEQ ID NO: 1 or SEQ ID NO: 2. Described in several exemplary antibodies herein are antibodies or fragments thereof comprising one or more polypeptides each independently having a sequence according that is 80%-100% identical SEQ ID NO: 1 or SEQ ID NO: 2, such as 80%, to/or 81%, 82%, 83%, 84%, 85%, 86%, 87%, 88%, 89%, 90%, 91%, 92%, 93%, 94%, 95%, 96%, 97%, 98%, 99%, 100% identical SEQ ID NO: 1 or SEQ ID NO: 2. In some embodiments the antibody is a monoclonal antibody. In some embodiments the antibody is a polyclonal antibody. Without being bound by theory, the antibodies and fragments thereof described herein can be useful in methods of detecting and/or treating B. burgdorferi infection as described elsewhere herein.

In some embodiments, the antibody or fragment thereof is capable of specifically binding a B. burgdorferi-specific peptidoglycan or fragment thereof. In some embodiments, the antibody or fragment thereof is capable of specifically binding B. burgdorferi-specific peptidoglycan or fragment comprising a GlcNAc-GlcNAc-MurNAc (GGM) trisaccharide.

### Encoding Polynucleotides

Described in several example embodiments herein are engineered polynucleotide(s) that encode a polypeptide having a sequence according to SEQ ID NO: 1 or SEQ ID NO: 2. Described in several example embodiments herein are engineered polynucleotide(s) that encode a polypeptide having a sequence according that is 80%-100% identical SEQ ID NO: 1 or SEQ ID NO: 2, such as 80%, to/or 81%, 82%, 83%, 84%, 85%, 86%, 87%, 88%, 89%, 90%, 91%, 92%, 93%, 94%, 95%, 96%, 97%, 98%, 99%, 100% identical SEQ ID NO: 1 or SEQ ID NO: 2. In some embodiments, the engineered polynucleotide is codon optimized for expression in specific cell or tissue type. In some embodiments, the engineered polynucleotide is codon optimized for expression in specific species or organism. Codon optimization is described in further detail elsewhere herein. The engineered polynucleotides can be DNA or RNA or DNA/RNA hybrids.

### Vectors and Vector Systems

Also provided herein are vectors that can contain one or more of the engineered polynucleotides described herein, such as those encoding an antibody or fragment thereof of the present disclosure. In some embodiments, one or more of the engineered polynucleotides(s) are operatively coupled to one or more regulatory elements, such as promoters. The vectors can be useful in producing bacterial, fungal, yeast, plant cells, animal cells, and transgenic animals that can express and/or produce one or more antibodies or fragments thereof of the present disclosure. The vectors and/or vector systems can be used, for example, to express one or more of the polynucleotides in a cell, such as a producer cell, to produce virus particles described elsewhere herein or to produce antibodies or fragments thereof. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of an antibody or fragment thereof in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited, to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more of the engineered polynucleotides of the present disclosure so as to drive expression of the of the engineered polynucleotides of the present disclosure.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

### Cell-Free Vector and Polynucleotide Expression

In some embodiments, the polynucleotide encoding an antibody or fragment thereof of the present disclosure is expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg²⁺, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E. coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the engineered polynucleotides and/or vectors thereof described herein can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-la, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Ferl14), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the engineered polynucleotide(s) can be placed under control of a plant promoter, i.e., a promoter operable in plant cells. The use of different types of promoters is envisaged. A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use for expression of the engineered polynucleotide(s) are found in Kawamata et al., (1997) Plant Cell Physiol 38:792-803; Yamamoto et al., (1997) Plant J 12:255-65; Hire et al, (1992) Plant Mol Biol 20:207-18,Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include an engineered polynucleotide of the present disclosure a light-responsive cytochrome heterodimer (e.g. from Arabidopsis thaliana), and a transcriptional activation/repression domain. In some embodiments, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

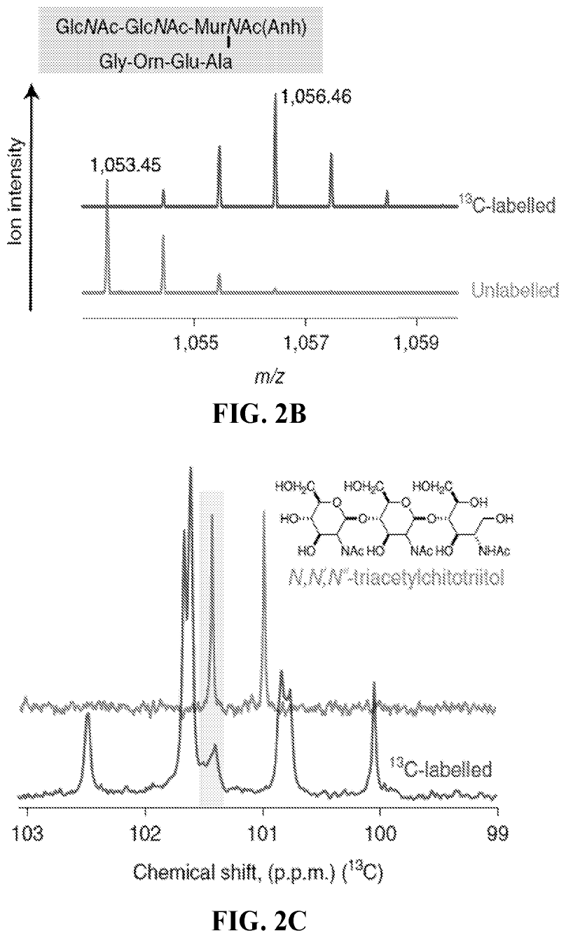

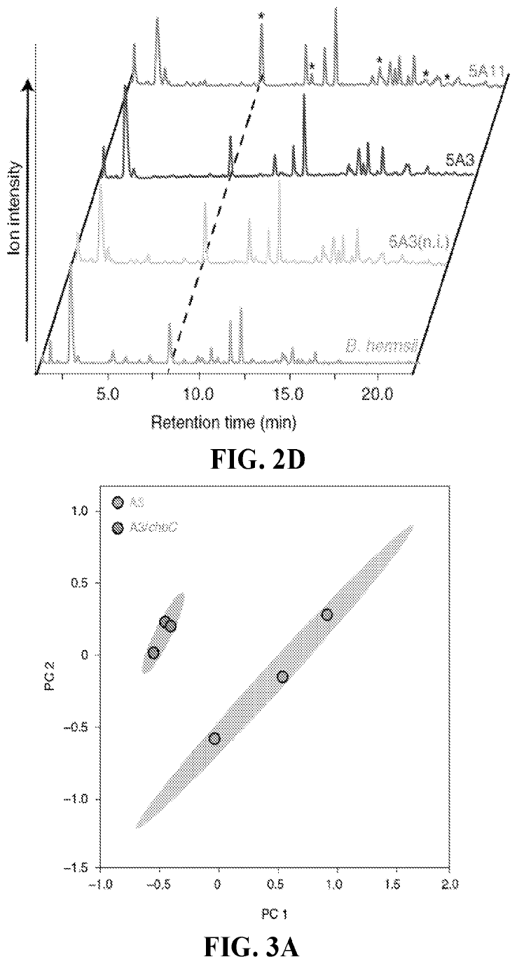

In some embodiments, the polynucleotide, vector or system thereof can include one or more elements capable of translocating and/or expressing an engineered polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/ and Negi et al., 2015. Database. 2015: bav003; doi: 10.1093/database/bav003), nuclear export signals (e.g., LXXXLXXLXL (SEQ ID NO: 3) and others described elsewhere herein), endoplasmic reticulum localization/retention signals (e.g., KDEL, KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http:minimotifminer.org, http://mitominer.mrc-mbu.cam.ac.uk/release-4.0/embodiment.do?name=Protein %20MTS), LocDB (see above), PTSs predictor ( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the engineered polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide such that the selectable marker polypeptide, when translated, is inserted between two amino acids between the N- and C-terminus of the antibody or fragment thereof of the present disclosure or at the N- and/or C-terminus of the antibody or fragment thereof of the present disclosure. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the antibody or fragment thereof of the present disclosure in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as P-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the CRISPR-Cas system described herein via suitable linker, such as a glycine or glycine serine linkers as short as GS or GG up to (GGGGG)₃ (SEQ ID NO: 4) or (GGGGS)₃ (SEQ ID NO: 5). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered polynucleotide(s) and/or products expressed therefrom include the targeting moiety and can be targeted to specific cells, tissues, organs, etc. In some embodiments, such as non-viral carriers, the targeting moiety can be attached to the carrier (e.g. polymer, lipid, inorganic molecule etc.) and can be capable of targeting the carrier and any attached or associated engineered polynucleotide(s) and/or antibodies or fragments thereof of the present disclosure to specific cells, tissues, organs, etc.

### Codon Optimization of Polynucleotides and Vector Polynucleotides

As described elsewhere herein, the engineered polynucleotide(s) encoding one or more antibodies or fragments thereof of the present disclosure can be codon optimized. In some embodiments, one or more polynucleotides contained in a vector (“vector polynucleotides”) described herein that are in addition to an optionally codon optimized engineered polynucleotide encoding an antibody or fragment thereof of the present disclosure can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered polynucleotide of the present invention, into a virus particle and producing said virus particle when used alone or with one or more other viral vectors (such as in a viral vector system). Viral vectors and systems thereof can be used for producing viral particles for delivery of and/or expression of one or more antibodies or fragments thereof described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

The systems and compositions of the present disclosure may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Methods and techniques for virus production from viral vectors is generally known in the art.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating the engineered polynucleotide(s) and delivering said engineered polynucleotide(s) to a cell and/or expressing the polynucleotide in the cell. It will be appreciated that this does not exclude vectors containing a polynucleotide designed to target a virus-based polynucleotide that is to be delivered.

### Naked Polynucleotides

In some embodiments one or more engineered polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered polynucleotide(s) of the present invention. In some embodiments, the naked polynucleotide can contain other nucleic acids and/or polynucleotides in addition to the engineered polynucleotide(s) of the present invention. The naked polynucleotides can include one or more elements of a transposon system. Transposons and systems thereof are described in greater detail elsewhere herein.

### Non-Viral Polynucleotide Vectors

In some embodiments, one or more of the engineered polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more CRISPR-Cas system polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Non-Vector Delivery Vehicles

The engineered polynucleotides, vectors, and/or antibodies and/or fragments thereof of the present disclosure can be delivered, such as to a cell or subject, via a non-vector delivery vehicle. The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations. In some examples. LNPs may be used for delivering DNA molecules and/or RNA molecules.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, Dec. 2011).

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan 31. pii: 50168-3659(17)30038-X. doi: 10.1016/j.jconrel.2017.01.037. [Epub ahead of print]; Altmoǧlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, Sep. 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (Jan. 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316;

### Liposomes

In some embodiments, a lipid delivery particle may be liposome. Liposomes are substantially spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the CRISPR-Cas systems described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs are as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2ρ (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Østergaard et al., Bioconjugate Chem., 2015, 26 (8), pp 1451-1455;

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the antibody or fragment thereof directly and delivered to cells or a subject.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the engineered polynucleotide. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos, e.g., engineered polynucleotides and/or antibodies or fragments thereof of the present disclosure. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, and US 20080267903.

### Streptolysin O (SLO)

The delivery vehicles can be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr 28. doi: 10.1039/dObm00427h.

Other non-limiting, exemplary exosomes include any of those set forth in Alvarez-Erviti et al. 2011, Nat Biotechnol 29: 341; [1401]El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Cells and Organisms

Described in certain example embodiments herein are engineered cells that can include an engineered polynucleotide of the present disclosure, a vector or vector system of the present disclosure, an antibody or fragment thereof of the present disclosure, or any combination thereof. In some embodiments, the cells are capable of production of an antibody or fragment thereof of the present disclosure. In some embodiments, the cells are capable of production of an antibody or fragment thereof of the present disclosure at an industrial scale.

In some embodiments, the cells are eukaryotic cells. In some embodiments, the cells are prokaryotic cells. In some embodiments, the cells are fungal cells, insect cells, plant cells, or animal cells. In some embodiments, the cells are human cells. In some embodiments, the cells are mammalian cells. In some embodiments, the cells are avian cells. In some embodiments, the cells are murine, bovine, ovine, porcine, equine, canine, feline, cervine, and/or the like.

Described in several exemplary embodiments herein are engineered organisms comprising one or more of the engineered cells described herein. In some embodiments, the engineered organisms are plants. In some embodiments, the engineered organisms are non-human animals. In some embodiments, the engineered organisms are non-human mammals. Exemplary non-human mammals include, without limitation, murine, bovine, ovine, porcine, equine, canine, feline, cervine, and/or the like. In some embodiments, the engineered organisms are avian. The engineered organisms can be used as bioreactors to produce the antibodies and/or fragments thereof of the present invention. The engineered organisms can produced the antibodies or fragments thereof in one or more bodily fluids or plant part. The antibodies and/or fragments thereof can be purified from the bodily fluids and/or plant part. Methods and techniques for generating engineered organisms are generally known in the art.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. In some embodiments, the pharmaceutical formulation comprises an antibody of fragment thereof of the present disclosure as a primary or secondary active agent. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or any combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a Lyme's disease and/or B. burgdorferi infection, or a symptom thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combinations thereof.

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects are to treat or prevent infection with B. burgdorferi, treat or prevent Lyme's disease, or a symptom thereof.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰/cells/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulation. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In some embodiments, the co-therapy includes an antibody or fragment thereof of the present disclosure and an anti-infective, optionally where the anti-infective agent comprises doxycycline, amoxicillin, cefuroxime, cefotaxime, azlocillin, penicillin, erythromycin, ceftriaxone, or any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### KITS

Described in example embodiments herein are kits, such as combination kits, that contain one or more compounds, compositions, formulations, devices, for performing a method described herein. As used herein, the terms “combination kit” or “kit of parts” refers to the compounds, compositions, formulations, particles, devices, and any additional components that are used to collect samples, store samples, ship samples, test samples, package, sell, market, deliver, and/or administer an element, combination of elements or a single element, such as the active ingredient, contained therein. Such additional components include, but are not limited to, packaging, syringes, blister packages, bottles, solutions, detection reagents, swabs, collection vials, collection tubes, labels and the like.

In some embodiments, a kit includes one or more reagents capable of adapted for detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide.

In some embodiments, the kit includes (a) one or more reagents capable of or adapted for detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; (b) one or more compositions or pharmaceutical formulations capable of treating a B. burgdorferi infection or a symptom thereof, wherein the one or more compositions or pharmaceutical formulations comprise an anti-infective agent, an anti-inflammatory agent, an analgesic, an antibody or fragment thereof, or any combination thereof; or (c) both (a) and (b). In some embodiments, the antibody or fragment thereof comprises or consists of one or more polypeptides each independently having a sequence according to SEQ ID NO: 1 or SEQ ID NO: 2. In some embodiments, the one or more reagents capable of or adapted for detecting comprises or consists of an antibody or fragment thereof, wherein the antibody or fragment thereof comprises or consists of one or more polypeptides each independently having a sequence according to SEQ ID NO: 1 or SEQ ID NO: 2. In some embodiments, the anti-infective agent comprises doxycycline, amoxicillin, cefuroxime, cefotaxime, azlocillin, penicillin, erythromycin, ceftriaxone, or any combination thereof.

In some embodiments, the combination kit also includes instructions printed on or otherwise contained in a tangible medium of expression. The instructions can provide information regarding the content of the compounds, compositions, formulations, particles, and/or devices contained in the kit, safety information regarding the content of the compounds, compositions, formulations, particles, and/or devices contained in the kit information regarding the dosages, indications for use, and/or recommended treatment regimen(s) for the compounds, compositions, formulations, particles, and/or devices contained in the kit. In some embodiments, the instructions can provide directions for administering or otherwise using the compounds, compositions, formulations, particles, and devices described herein or a combination thereof to a subject in need thereof. In some embodiments, the subject in need thereof is in need of diagnosing, prognosing, monitoring, staging, and/or treating Lyme's disease and/or a B. burgdorferi infection.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—B. burgdorferi Cell Wall Peptidoglycans

Peptidoglycan—a mesh sac of glycans that are linked by peptides—is the main component of bacterial cell walls. Peptidoglycan provides structural strength, protects cells from osmotic pressure and contributes to shape. All bacterial glycans are repeating disaccharides of N-acetylglucosamine (GlcNAc) β-(1-4)-linked to N-acetylmuramic acid (MurNAc). Borrelia burgdorferi, the tick-borne Lyme disease pathogen, produces glycan chains in which MurNAc is occasionally replaced with an unknown sugar. Nuclear magnetic resonance, liquid chromatography-mass spectroscopy and genetic analyses show that B. burgdorferi pro-duces glycans that contain GlcNAc-GlcNAc. This unusual disaccharide is chitobiose, a component of its chitinous tick vector. Mutant bacteria that are auxotrophic for chitobiose have altered morphology, reduced motility and cell envelope defects that probably result from producing peptidoglycan that is stiffer than that in wild-type bacteria. Without being bound by theory Applicant proposes that the peptidoglycan of B. burgdorferi probably evolved by adaptation to obligate parasitization of a tick vector, resulting in a biophysical cell-wall alteration to withstand the atypical torque associated with twisting motility.

### Introduction

The he peptidoglycan sacculus protects the cytoplasmic contents of virtually all bacterial cells. Peptidoglycan architecture (rigid glycan strands, cross-linked by flexible peptides) is universal across bacterial taxa. Peptidoglycan glycans comprise a disaccharide repeat unit of N-acetylglucosamine (GlcNAc) and N-acetylmuramic acid (MurNAc). MurNAc provides a C3 lactyl moiety that anchors peptide assembly. Glycan chain lengths of six to hundreds of disaccharide repeats are terminated at the reducing-end anomeric position by a 1,6-anhydro-N-acetylmuramic acid (anhMurNAc) residue¹. Although alterations in peptidoglycan peptide chemistry occur across the bacterial domain, deviations from the β-(1-4)-linked GlcNAc-MurNAc disaccharide have not previously been reported.

The pathogenic spirochaete B. burgdorferi is estimated to cause more than 450,000 cases of Lyme disease each year, in the USA alone². On transmission via the bite of an infected Ixodes scapularis tick, B. burgdorferi, which is an obligate parasitic bacterium, causes a biphasic infection. An acute stage characterized by ‘flu-like’ symptoms is followed by a severe late stage that can involve multiple organ systems^{3,4}. Despite the public health burden posed by this ascending vector-borne disease, very little is known about what causes clinical symptoms.

B. burgdorferi lacks many of the classic virulence factors typically associated with invasive pathogens. One well-known feature, critical to B. burgdorferi pathogenesis, is the corkscrew-like motility that it uses to both escape immune cells and invade host tissues⁵. Endoflagella at each pole form a ribbon that wraps around the peptidoglycan sacculus. Motor rotation causes the flagella to torque the peptidoglycan, creating a backward wave that propels the bacterium forwards⁶. B. burgdorferi peptidoglycan, which has also been implicated in potentiating Lyme disease pathogenesis^{7,8}, is thought to require unique feature(s) to counterbalance the immense flagellar stress. Previous reports describe the presence of ornithine (Orn) in the peptidoglycan stem peptide^{7,9}, as well as several unidentifiable components, including an unknown N-acetylated hexose (HexNAc) linked to the GlcNAc-MurNAc disaccharide in glycan strands⁷. The culprit responsible for this atypical alteration has remained unknown.

### Results

### B. Burgdorferi Glycan Architecture.

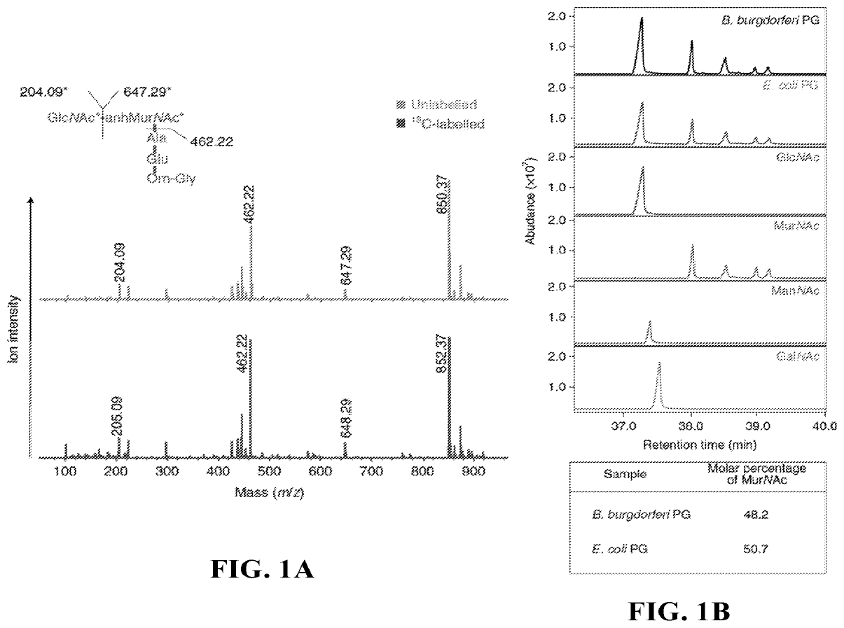

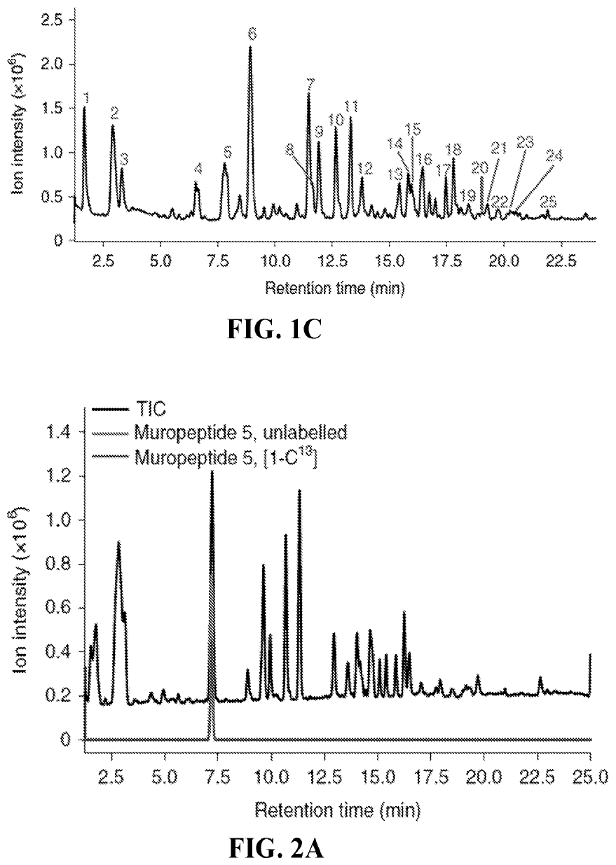

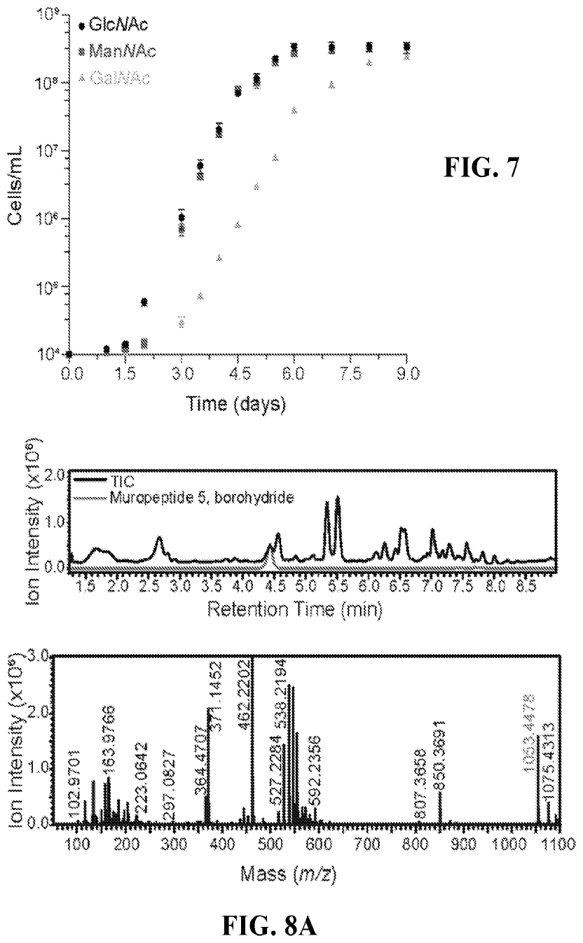

Similar to most parasitic bacteria, B. burgdorferi lacks many biosynthetic pathways and scavenges environmental molecules, including the peptidoglycan cell-wall precursor GlcNAc¹⁰. Optimal in vitro growth thus requires that B. burgdorferi culture medium be supplemented with GlcNAc¹¹. By taking advantage of this auxotrophy, we reasoned that we would be able to substitute GlcNAc with other N-acetylated sugars and identify the unknown hexose. Two candidates emerged for their ability to support growth in the absence of GlcNAc: N-acetylmannosamine (ManNAc) and N-acetylgalactosamine (GalNAc) (FIG. 7 and refs. 12-15). As bacteria cultured with ManNAc replicated at a similar rate and reached a comparable final density to GlcNAc (FIG. 7), we proceeded with metabolic labelling studies. Applicant propagated B. burgdorferi 5A11 in culture medium containing [1-¹³C]ManNAc and analysed the resulting muropeptide pool, obtained from purified and digested peptidoglycan, by liquid chromatography-mass spectroscopy (LC-MS) (FIG. 1A). Compared with muropeptide samples prepared from bacteria cultured with unlabelled GlcNAc (FIG. 1A), [1-¹³C]ManNAc-labelled muropeptides were identical and contained the expected mass shift, equally dis-tributed across both GlcNAc and MurNAc (FIG. 1A). This strongly suggested not only that are there pathway(s) capable of converting ManNAc, and probably GalNAc, to GlcNAc, but also that ManNAc was an unlikely candidate. Next, Applicant took a more holistic approach and performed monosaccharide analysis of purified peptidoglycan isolated from B. burgdorferi 5A11 and compared these results with various N-acetylated reference standards (FIG. 1B). Surprisingly, Applicant detected only GlcNAc and MurNAc, and the B. burgdorferi peptidoglycan sugar profile was identical to that of Escherichia coli. Collectively, the metabolic labelling studies and monosaccharide analysis suggested that the unknown HexNAc might be GlcNAc.

### Muropeptide Analysis of B. Burgdorferi Peptidoglycan.

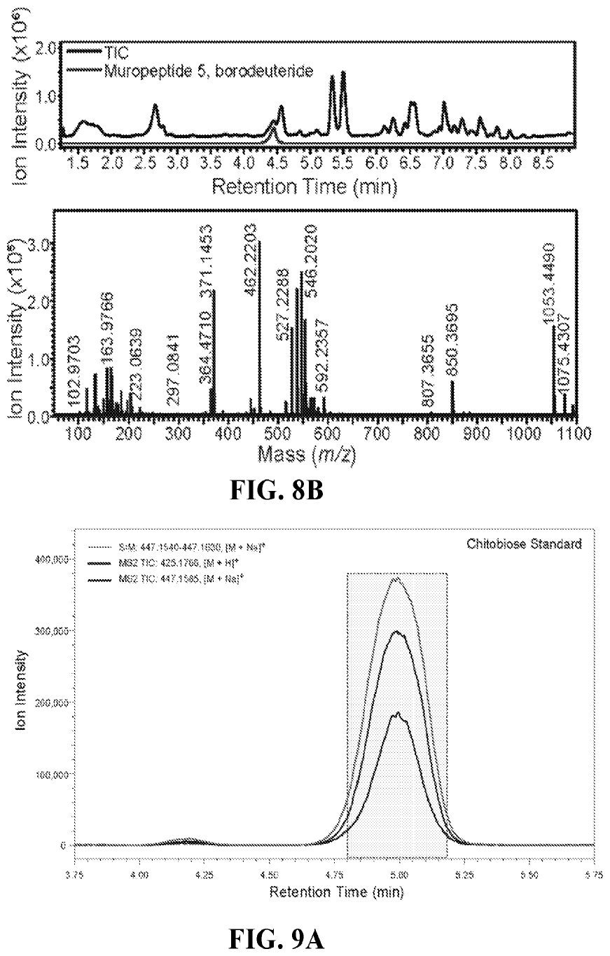

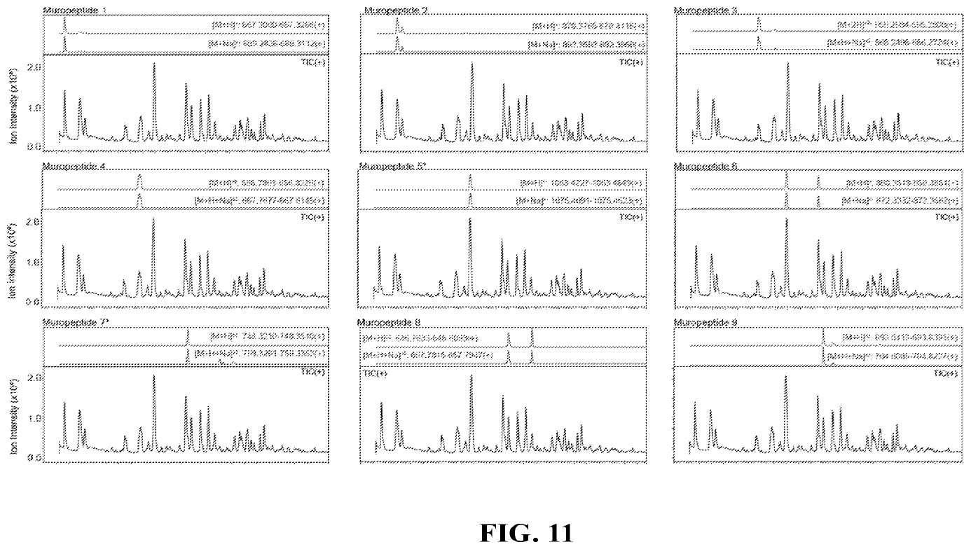

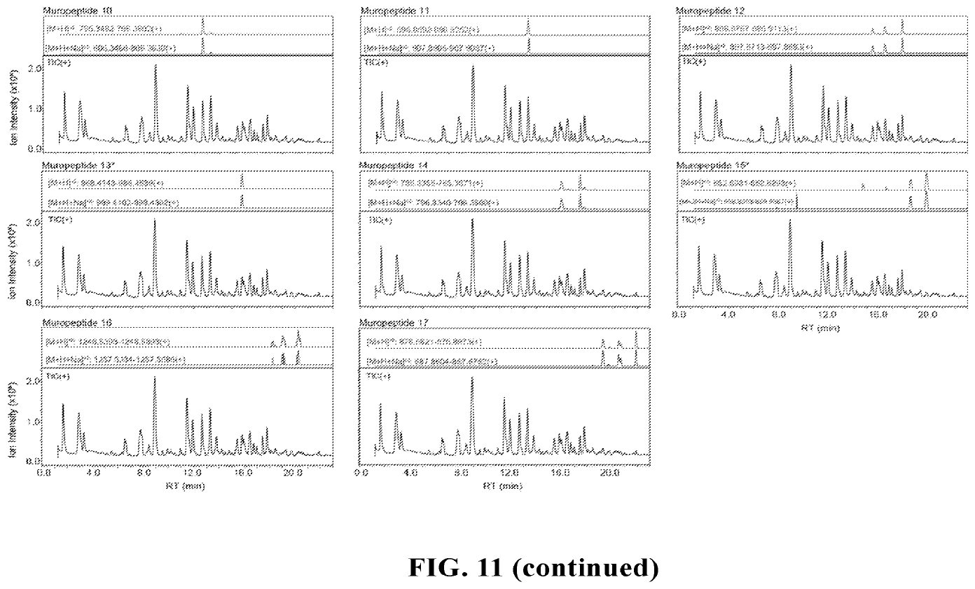

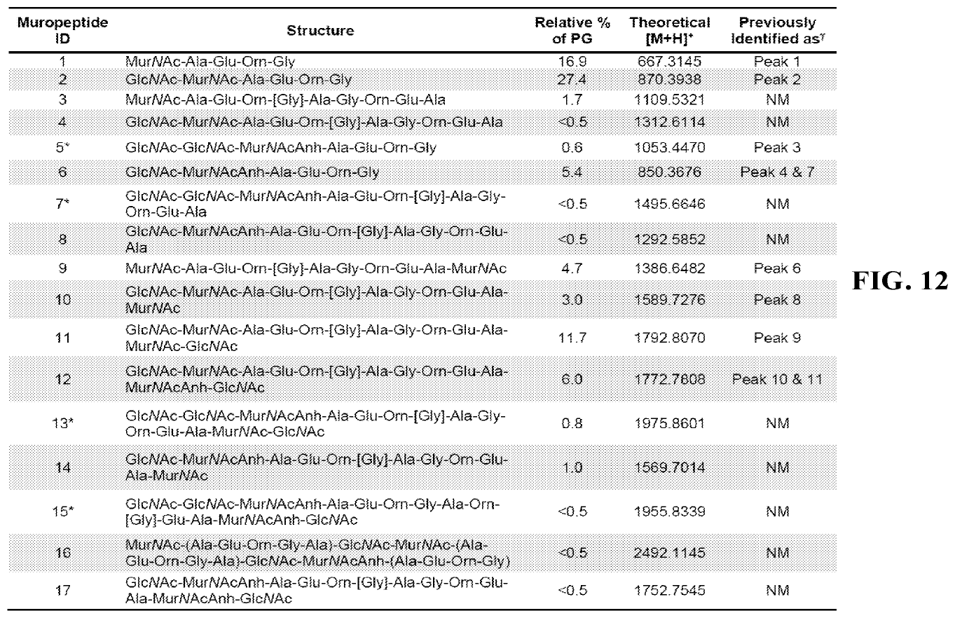

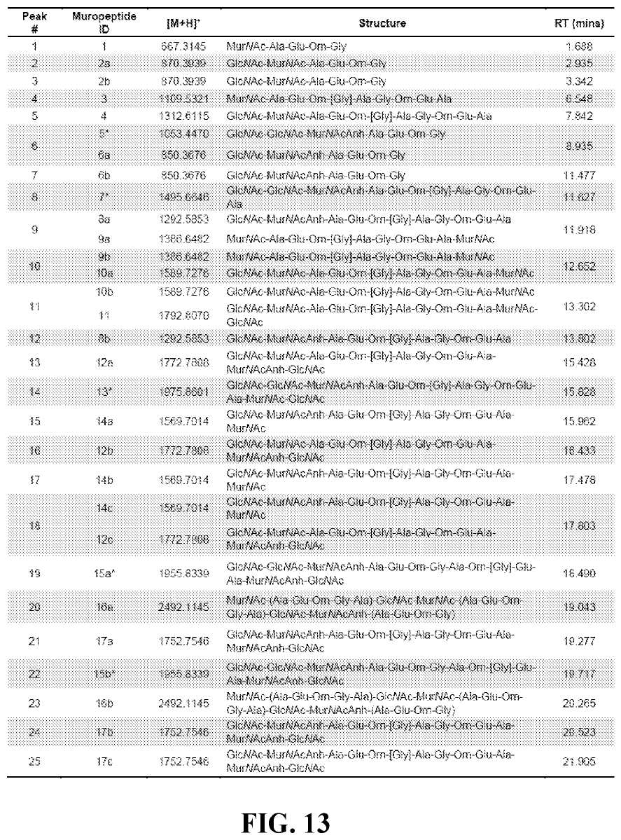

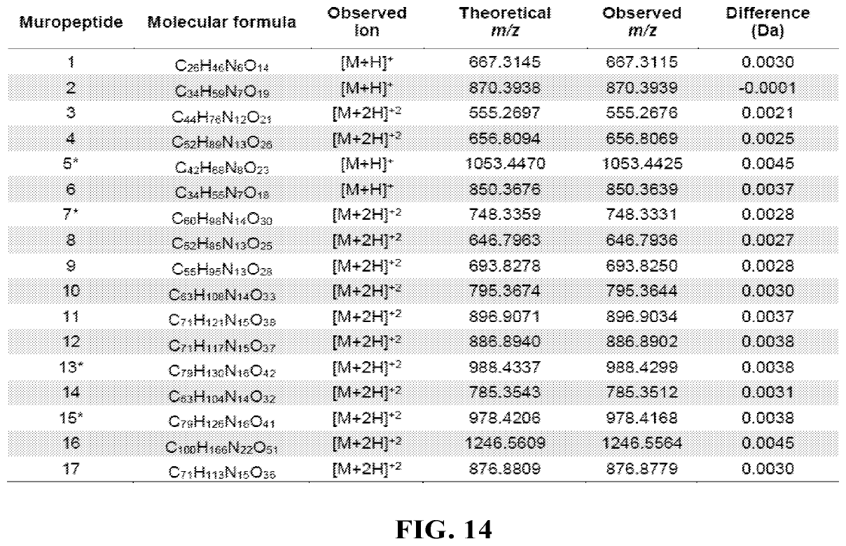

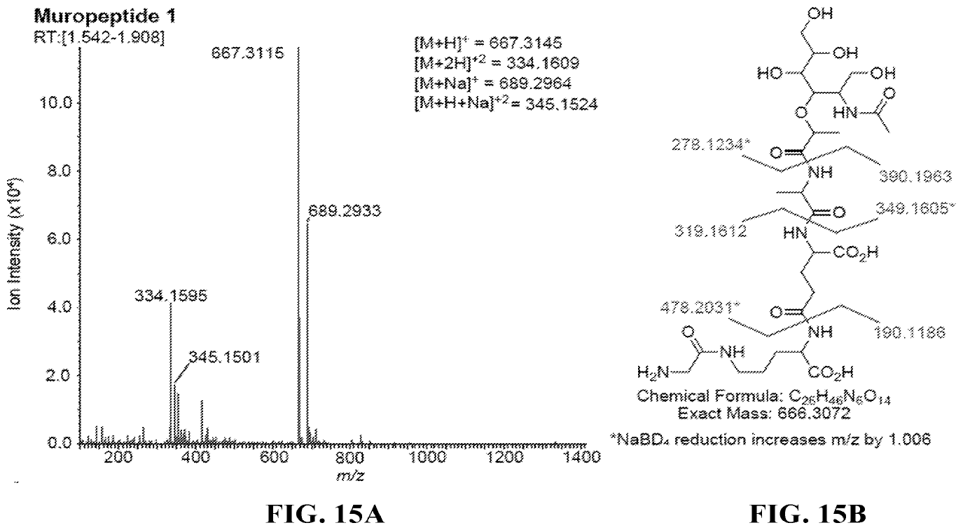

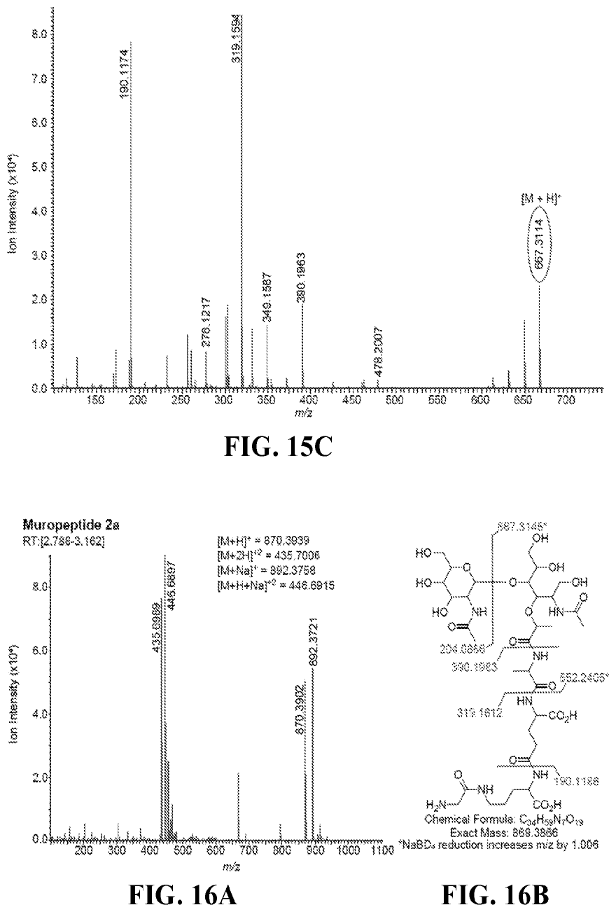

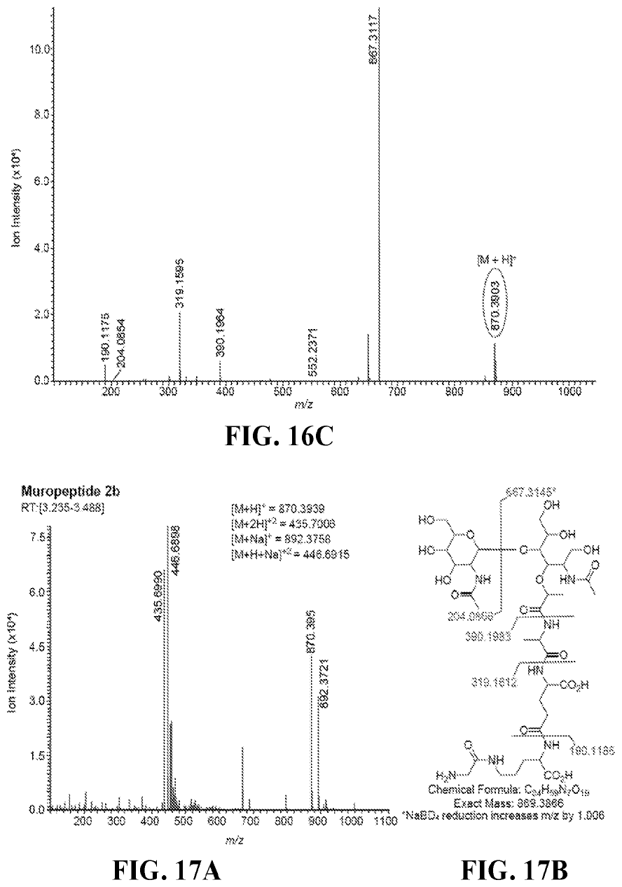

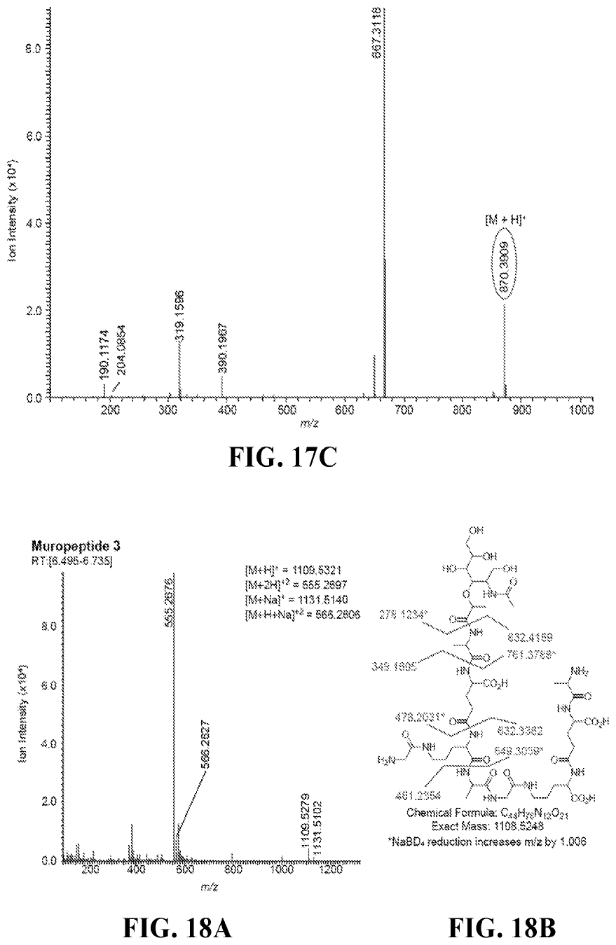

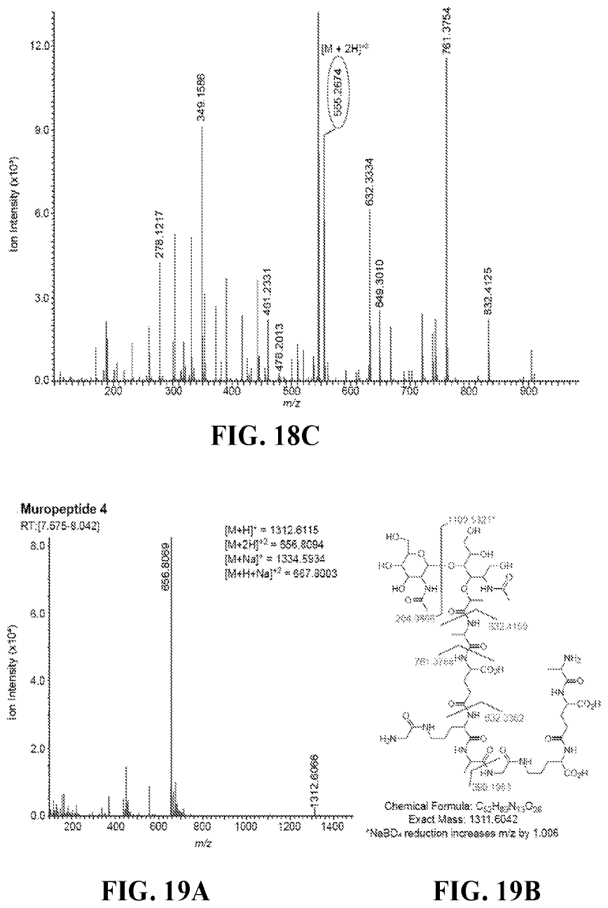

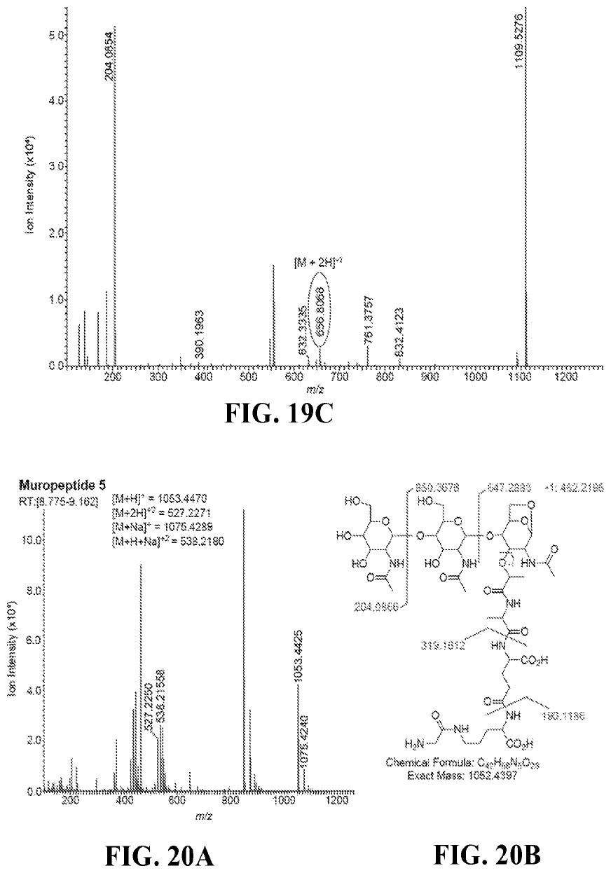

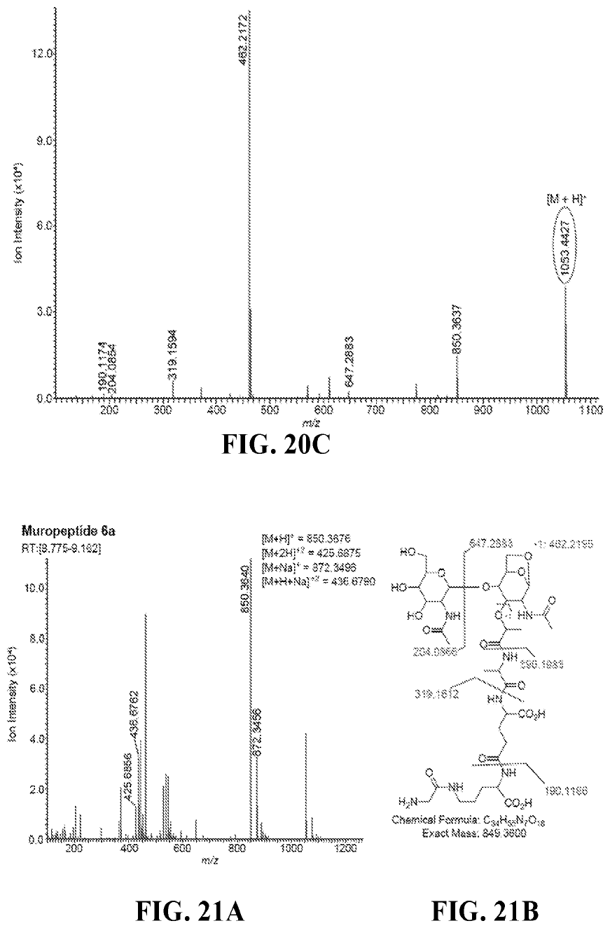

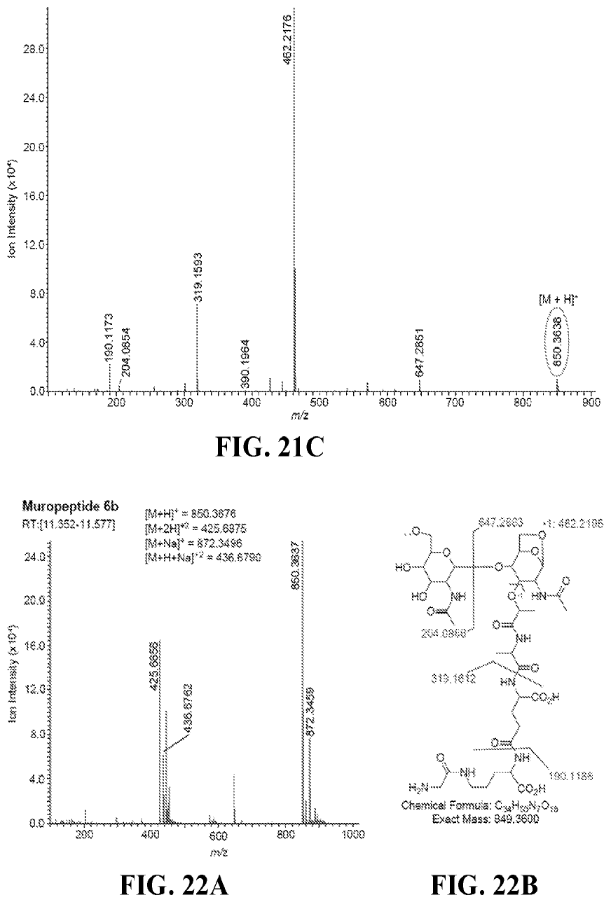

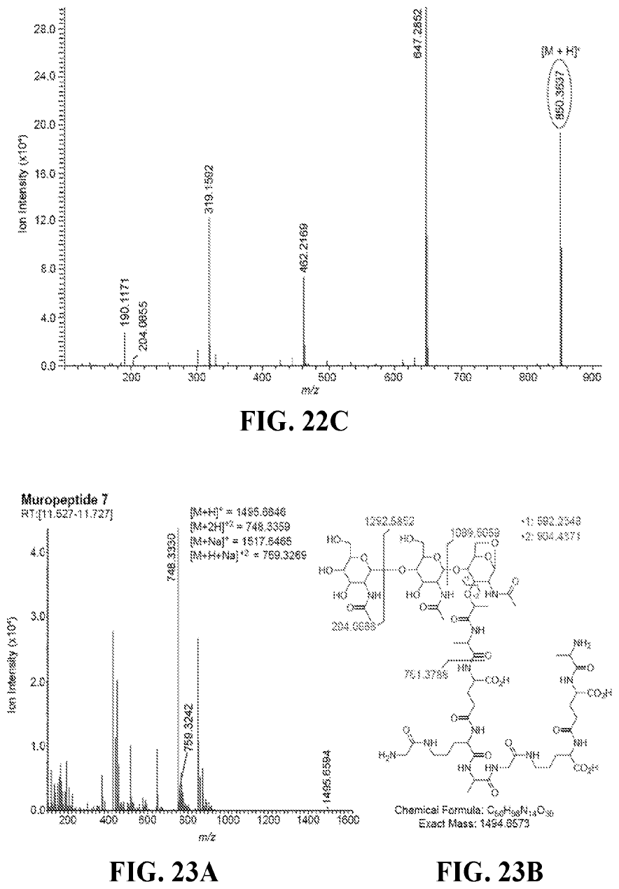

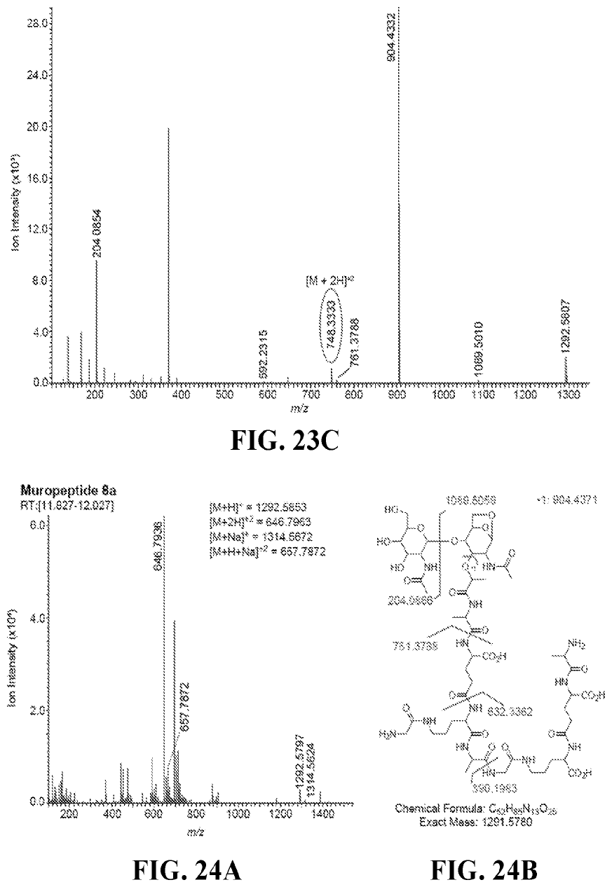

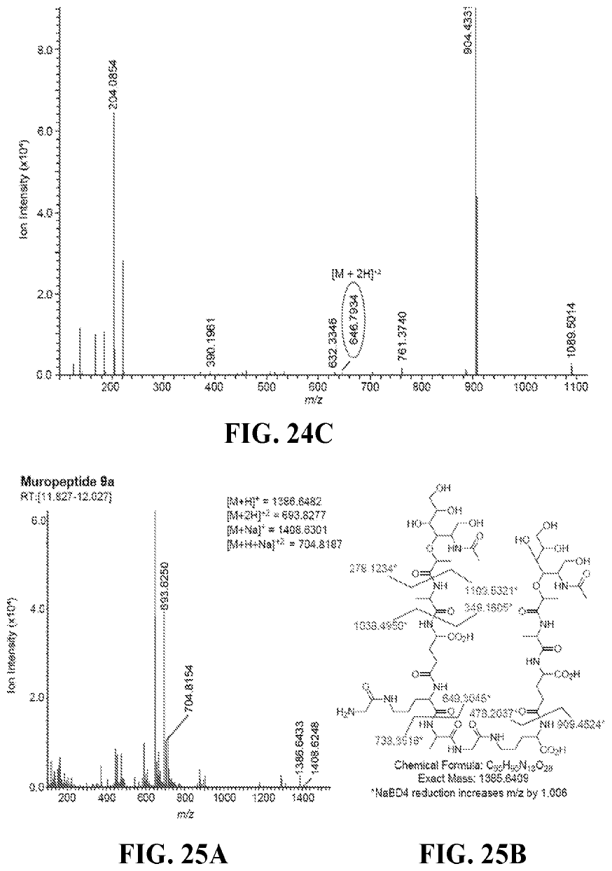

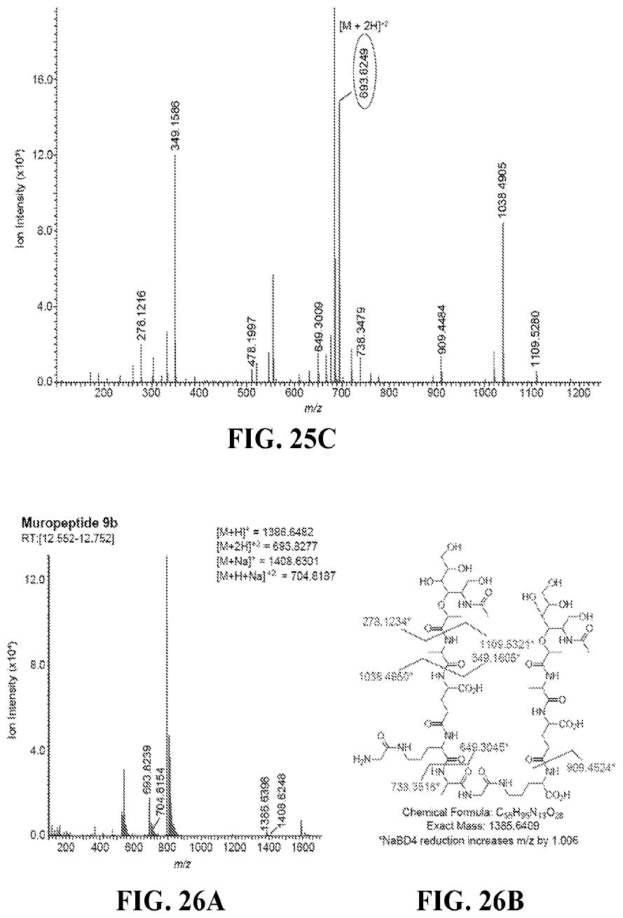

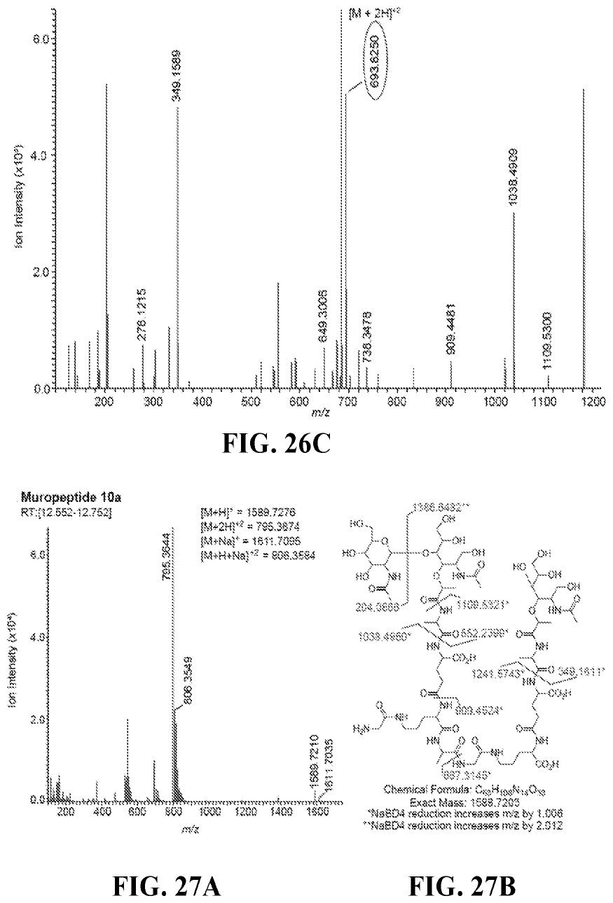

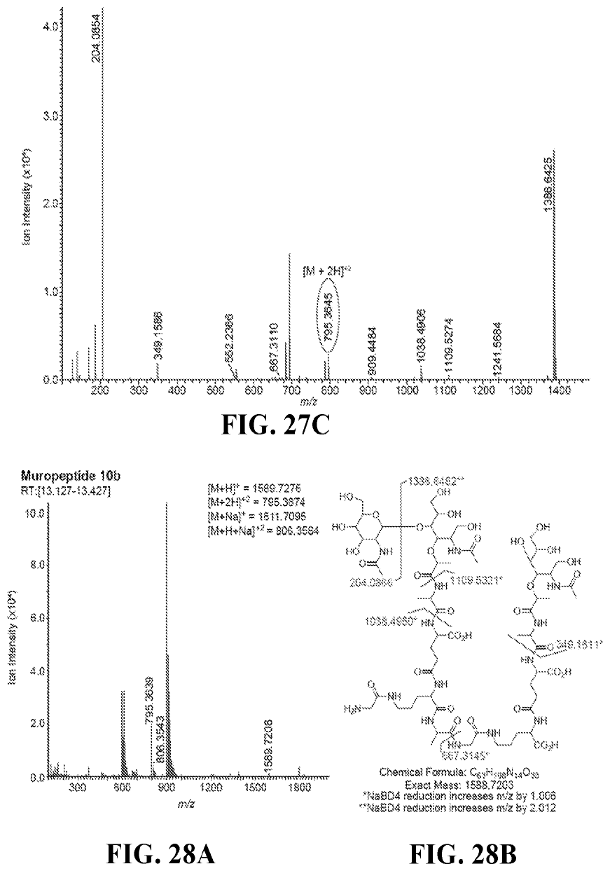

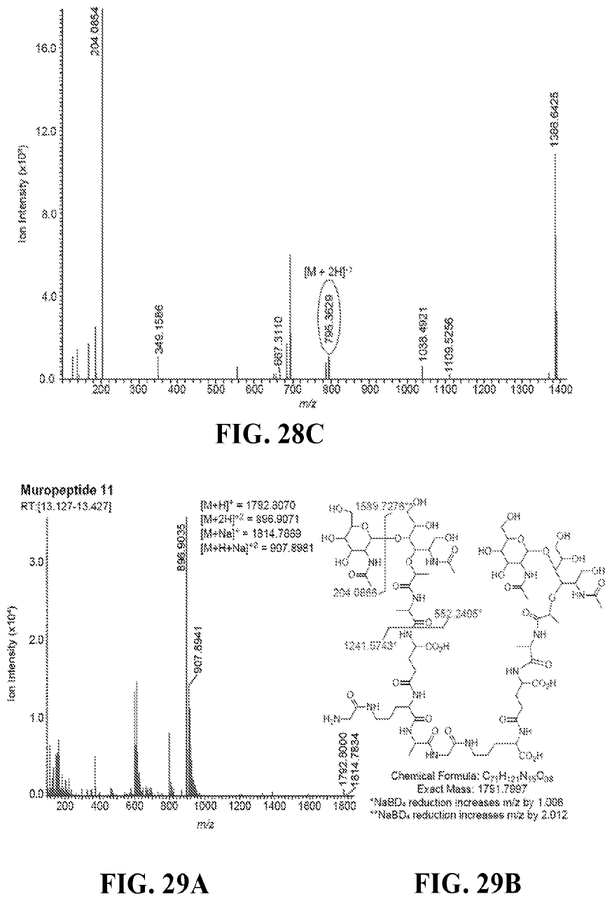

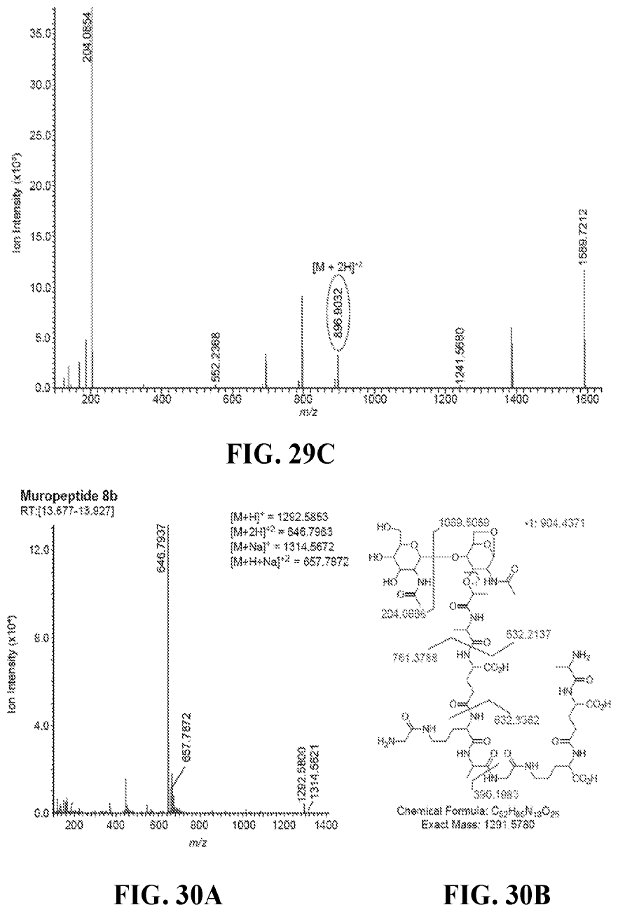

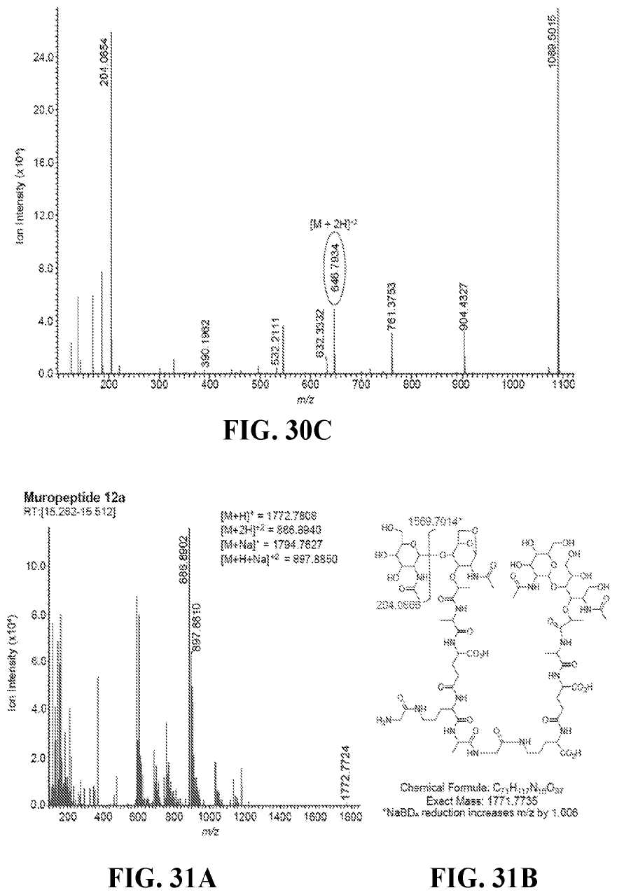

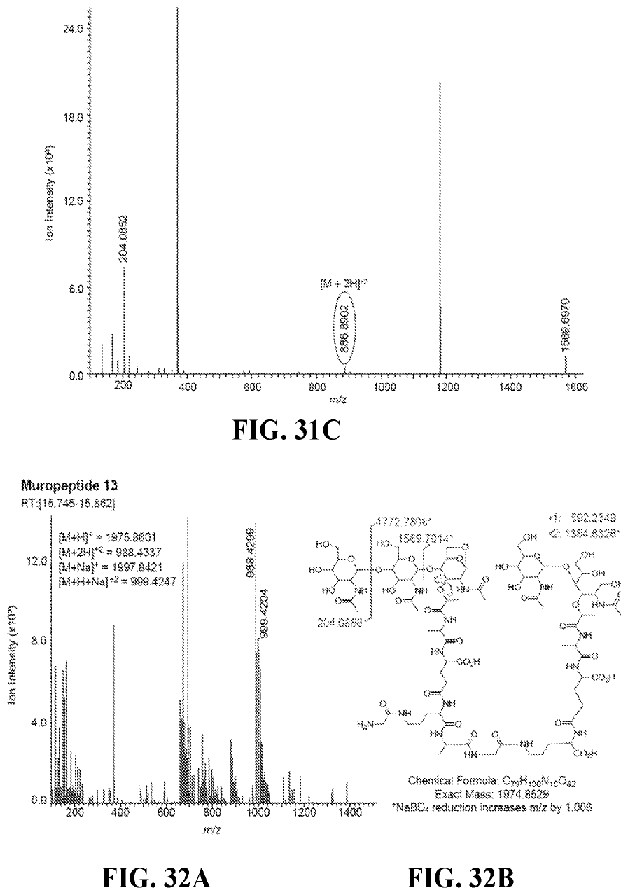

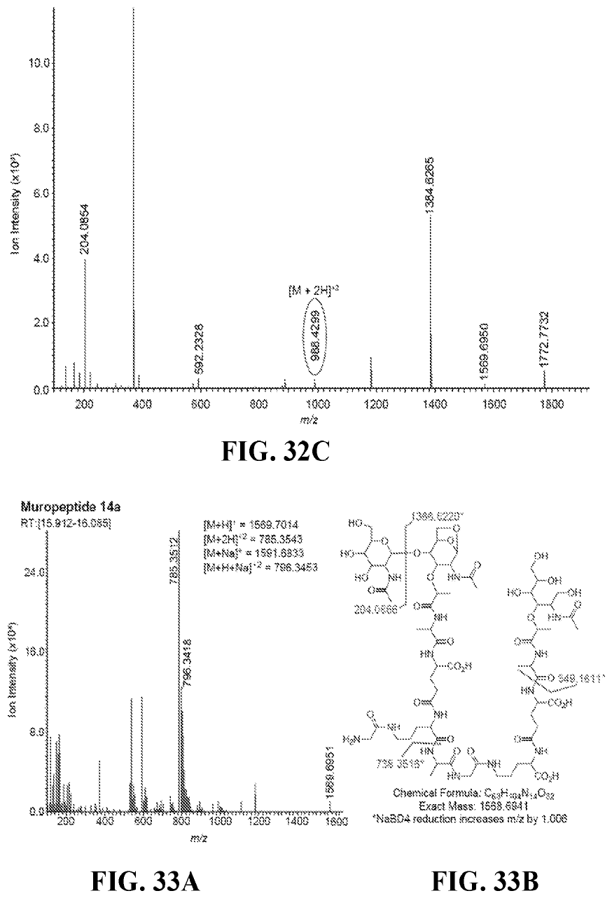

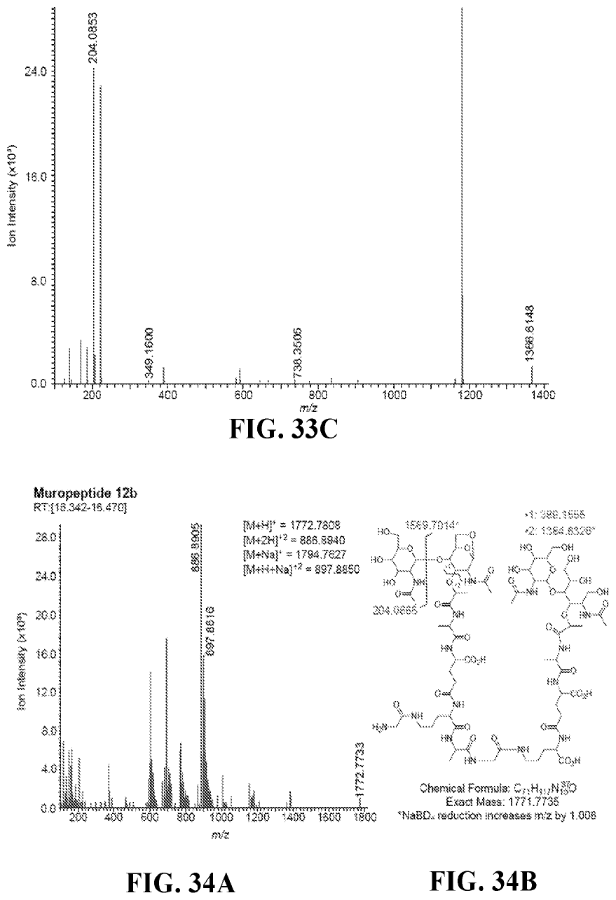

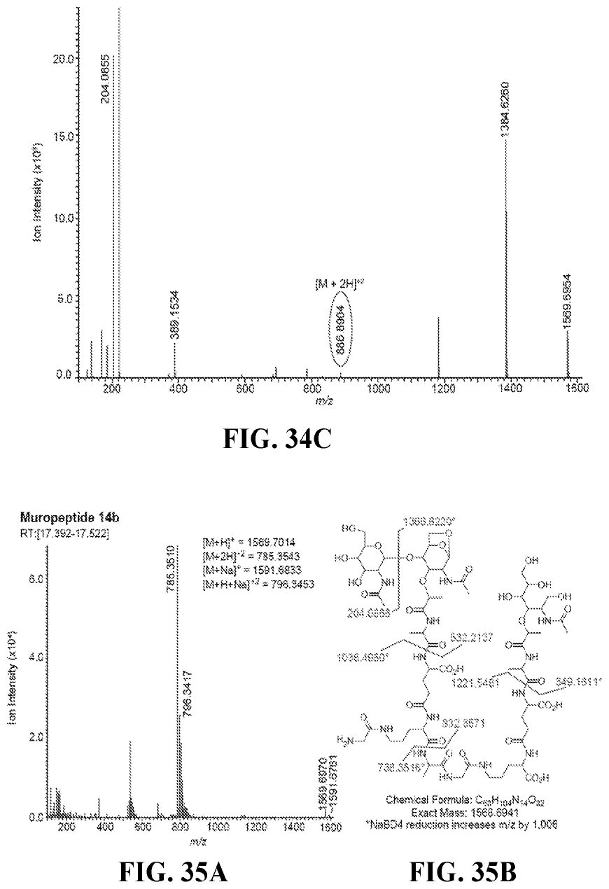

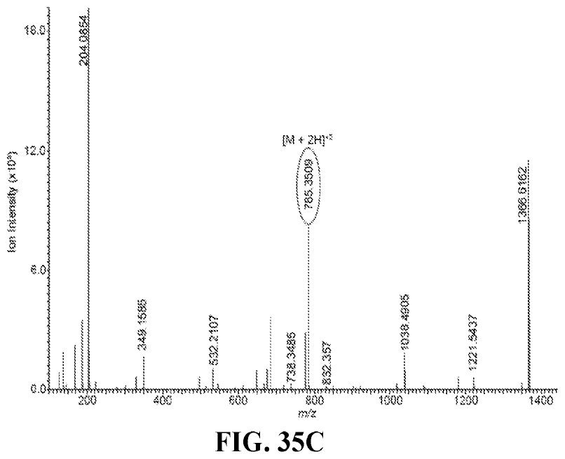

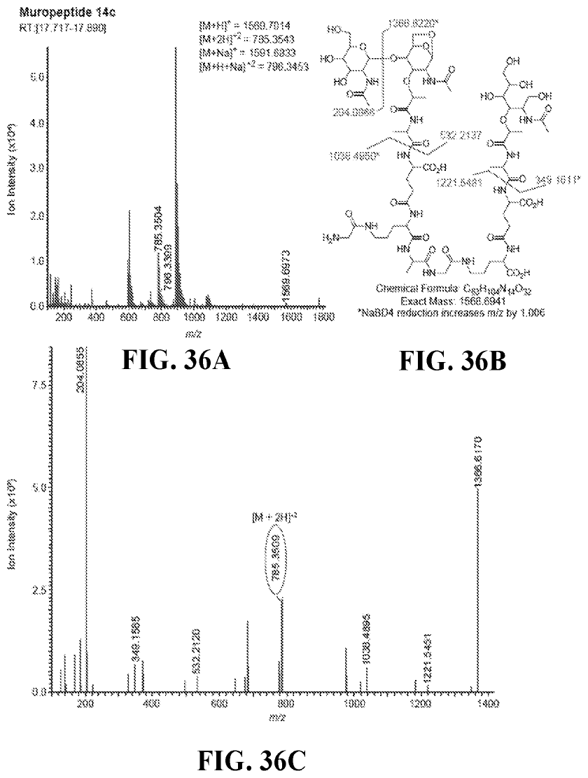

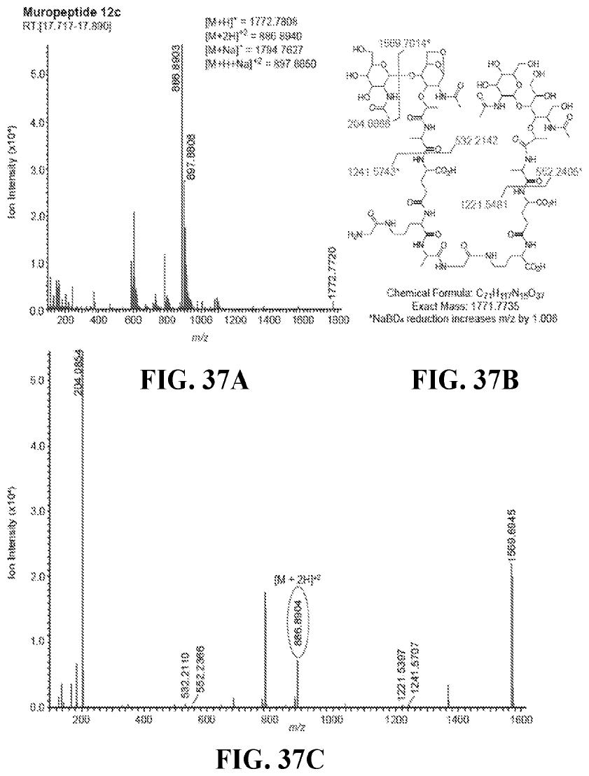

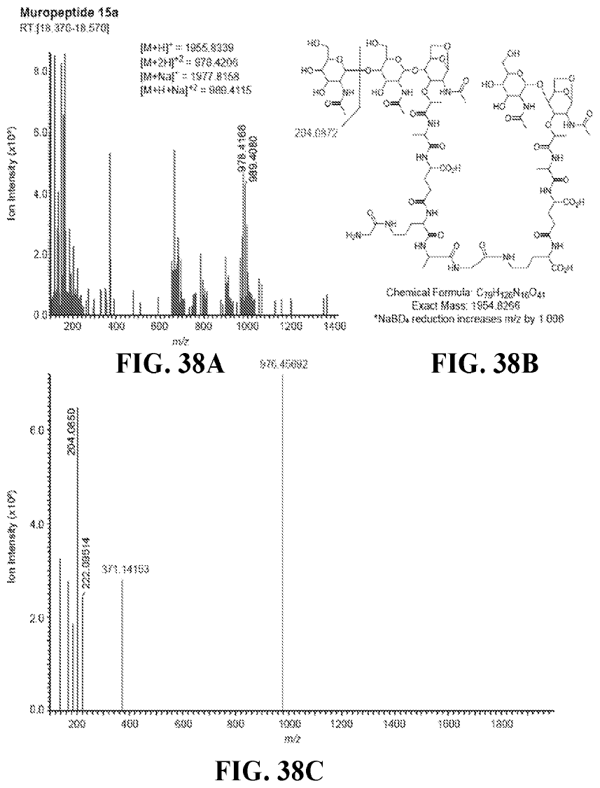

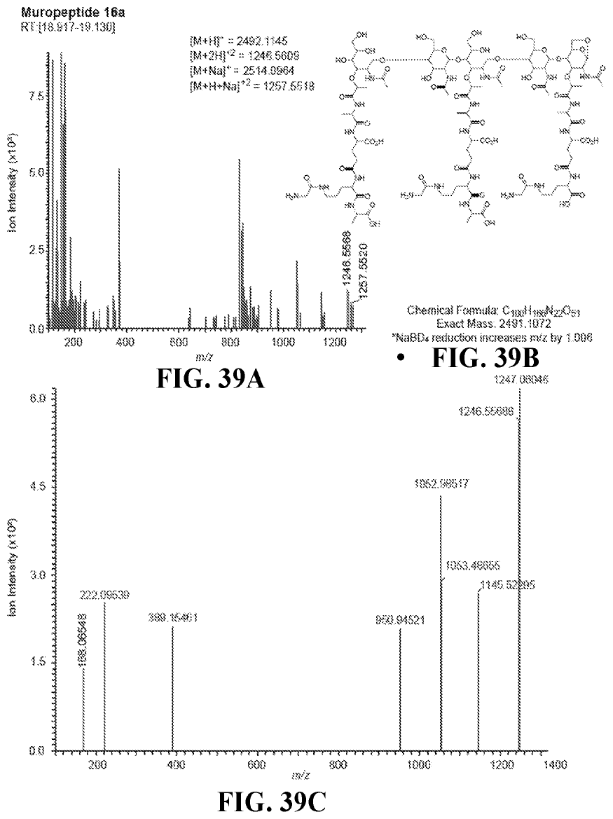

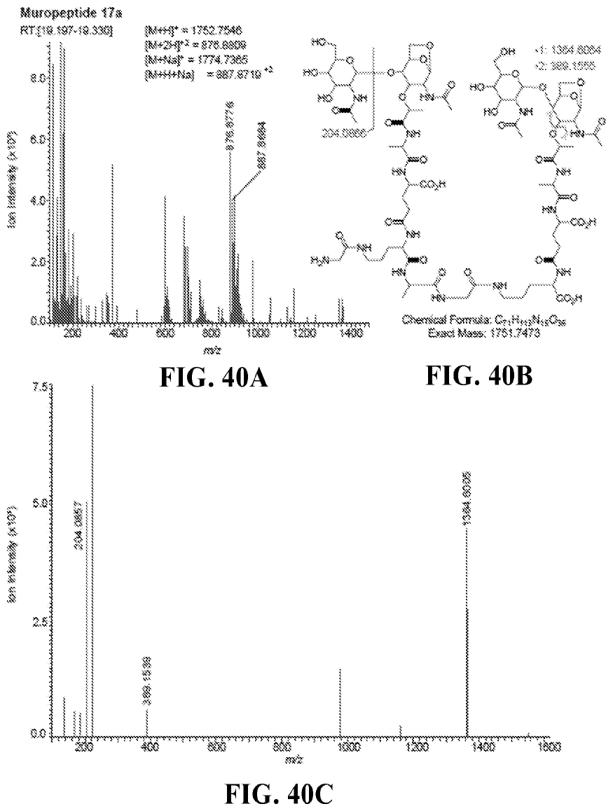

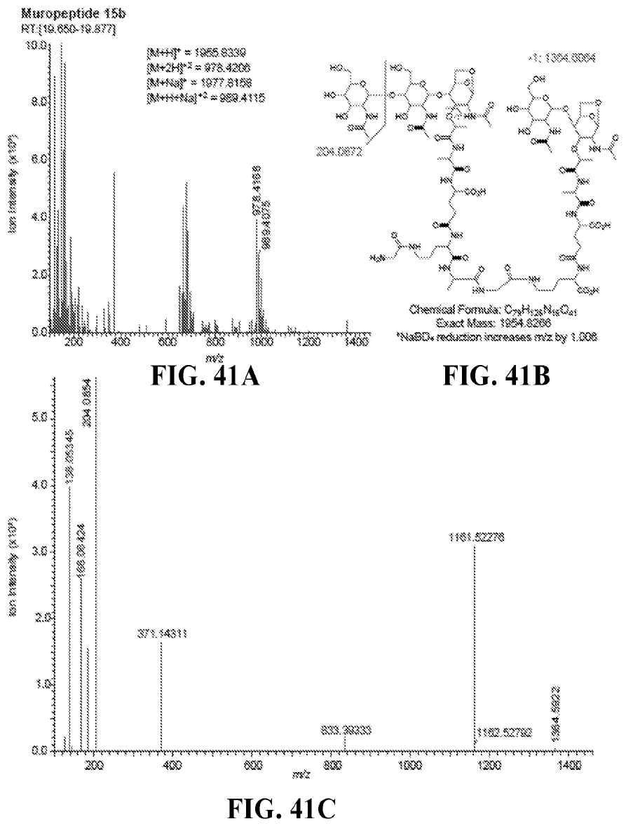

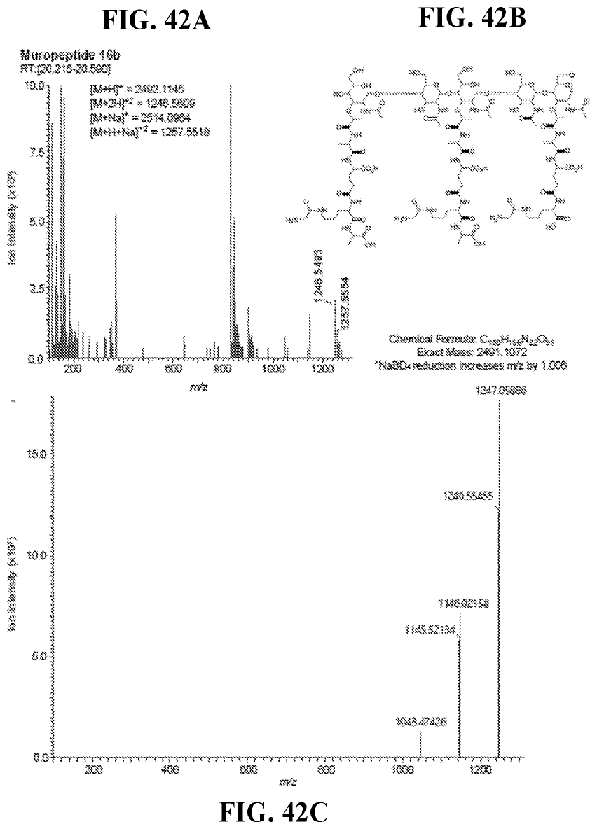

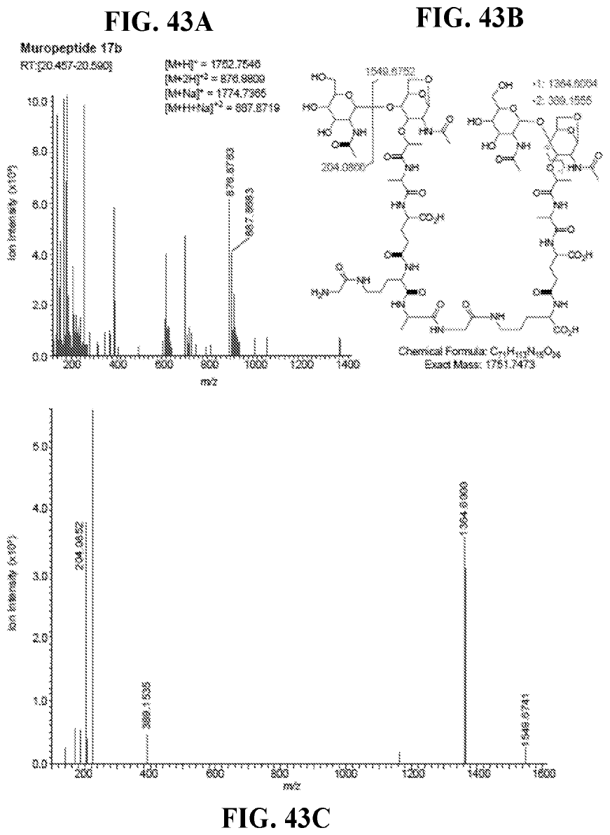

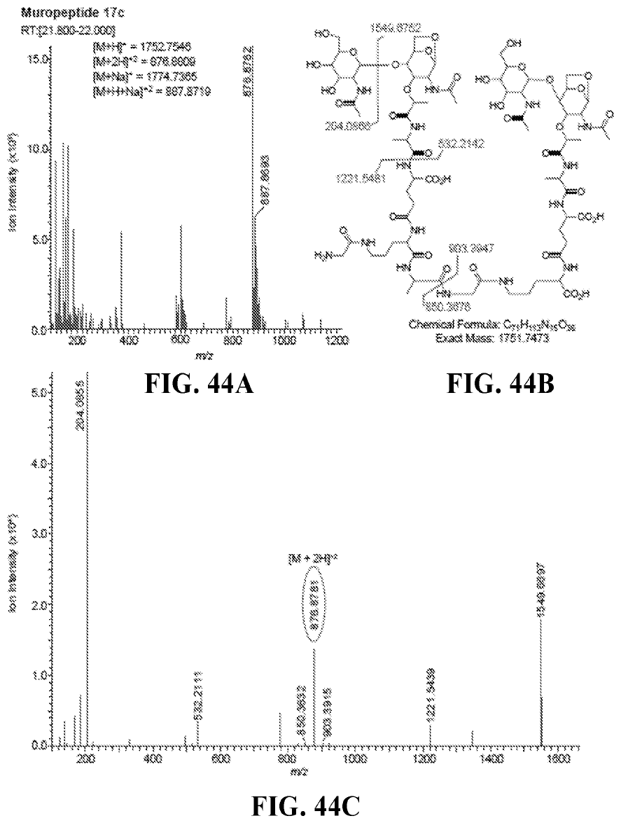

Previous analyses of the B. burgdorferi peptidoglycan cell wall separated muropeptides that were then analyzed using targeted MS. MS1 spectra for analyzed muropeptides are shown in FIGS. 15A-44C. This method captured the identity of ˜45% of the B. burgdorferi muropeptides⁷. Applicant reasoned that a more robust, untargeted approach to muropeptide analysis may provide further insights into composition of B. burgdorferi peptidoglycan. We created a new, high-resolution, LC-tandem MS (LC-MS/MS) method, which determined the identity of ˜80% of the muropeptide pool in a fraction of the time (FIG. 1C). The LC step separated 25 discrete peaks, which contained 17 unique muropeptides (FIG. 1C and FIG. 11), 5 of which contained the HexNAc-GlcNAc-MurNAc moiety (FIGS. 12-14, 20A-20C, 23A-23C, 32A-32C, 38A-38C, and 41A-41C). Applicant coupled LC-MS from NaBH4-reduced muropeptides with data obtained from isotopically (NaBD₄) labelled reduction products to provide mass markers and increased resolution for MS2 spectra in instances when more than one muropeptide eluted in the same fraction (FIG. 13 and FIG. 8A-8B). The latter confirmed that the unknown HexNAc was always adjacent to a GlcNAc-anhMurNAc residue, indicating that the new structure was at the terminus of glycan chains (FIG. 8A-8B) Regardless of whether B. burgdorferi was cultured in medium containing labelled ([1-¹³C]GlcNAc) or unlabelled GlcNAc, the resulting LC-MS traces of each sample were identical, with mass shifts confirming that the label was distributed between the GlcNAc and MurNAc residues (FIG. 2A-2B). These data further implicated GlcNAc as the unknown HexNAc because each sugar in the puta-tive GlcNAc-GlcNAc-anhMurNAc (G-G-anhM) trisaccharide was equally labelled (FIG. 2B).

Next, Applicant carried out a series of proton nuclear magnetic resonance (H-NMR) experiments using N,N′,N″-triacetylchitotriitol as a reference due to its structural similarity to G-G-anhM at the non-reducing end. As there are limitations associated with both purifying B. burgdorferi peptidoglycan and the detection limits for H-NMR, comparisons to the standard were in relation to the total muropeptide pool obtained from the [1-¹³C]GlcNAc experiment and not to an individual muropeptide. Anomeric 1H chemical shifts (>5 p.p.m.) and coupling constants (˜8 Hz) combined with ¹³C chemical shifts at ˜100 p.p.m. firmly established all linkages as P-glycopyranosidic bonds between GlcNAc residues (FIG. 2C). The only available hydroxyls for glycosidic bond formation are at positions 3, 4 and 6, with all known muropeptide linkages being (1-4)¹. Although we cannot exclude the existence of non-canonical (1-3) and glycosidic (1-6) bonds, the data that we obtained (FIG. 2C) match that of a 3-(1-4) linkage most closely. These findings establish that B. burgdorferi glycan chains terminate with G-G-anhM.

### Peptidoglycan Composition is Conserved Among Borrelia Strains and Species.

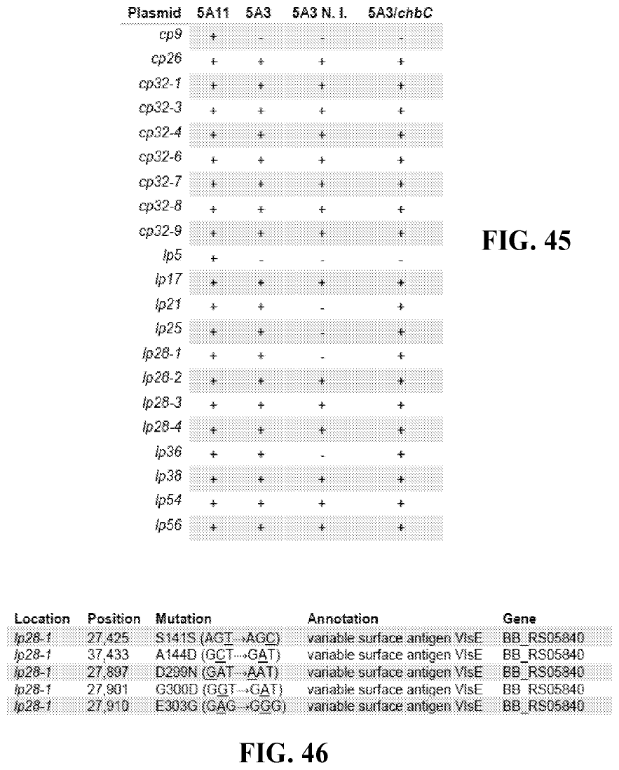

Laboratory strains of B. burgdorferi are known to lose extrachromosomal DNA during prolonged in vitro propagation¹⁶. This results in clonal heterogeneity, a reduction in biosynthetic capacity and avirulence¹⁶⁻¹⁸. To assess whether the peptidoglycan phenotype of B. burgdorferi 5A11 was due to a loss of extrachromosomal DNA, or an artefact of prolonged in vitro cultivation, Applicant used whole-genome sequencing (WGS) to analyze the three commonly studied strains of B31. Applicant sequenced strain 5A11, which is a fully infectious clone of the B31-type strain10, with all genetic elements that Applicant used for all the peptidoglycan work thus far, strain 5A3, a fully infectious clonal derivative16,18 of 5A11 that is often used in the Lyme disease research field, and a high-passage variant of B31 that lacks many plasmids and is avirulent (FIG. 45 and see data availability for repository links in DeHart et al., Nature Microbiol. 6:1583-1592. 2021.). WGS results were consistent with the expected nucleic acid content of each strain-5A11 and 5A3 were highly similar and carried a full repertoire of plasmids, whereas our high-passage strain lacked genetic elements associated with infectivity (FIG. 45). Upon strain validation Applicant isolated peptidoglycan from each and compared muropeptide profiles for the presence of G-G-anhM moieties. Each strain was almost identical and contained G-G-anhM (FIG. 2D).

Many different Borrelia genospecies cause Lyme disease. The analysis, thus far, has been limited to derivatives of the B31-type strain. Instead of testing various Lyme disease-causing Borrelia spp., applicant analyzed muropeptides from the relapsing fever pathogen B. hermsii, which is transmitted by Ornithodoros ticks¹⁹. Comparative analysis of muropeptide profiles, once again, clearly indicated the presence of G-G-anhM, despite differences in the abundance of other peptidoglycan fragments (FIG. 2D). Collectively, these studies demonstrate the first modification to the disaccharide repeat arrangement in bacterial glycans—a core biological feature of Borrelia peptidoglycan that is conserved, regardless of genome content or phylogenetic relatedness.

### Acquisition of GlcNAc-GlcNAc.

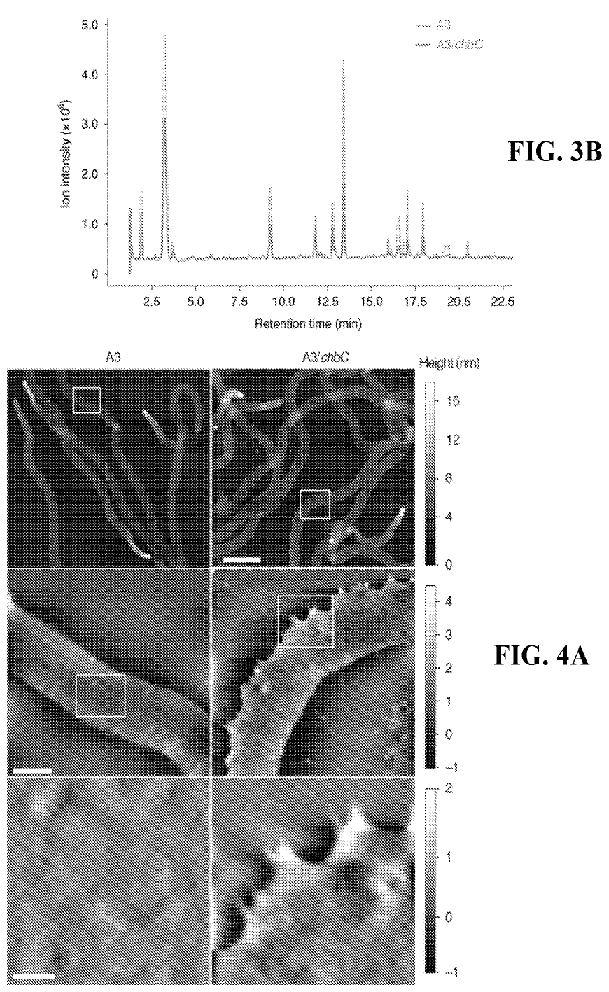

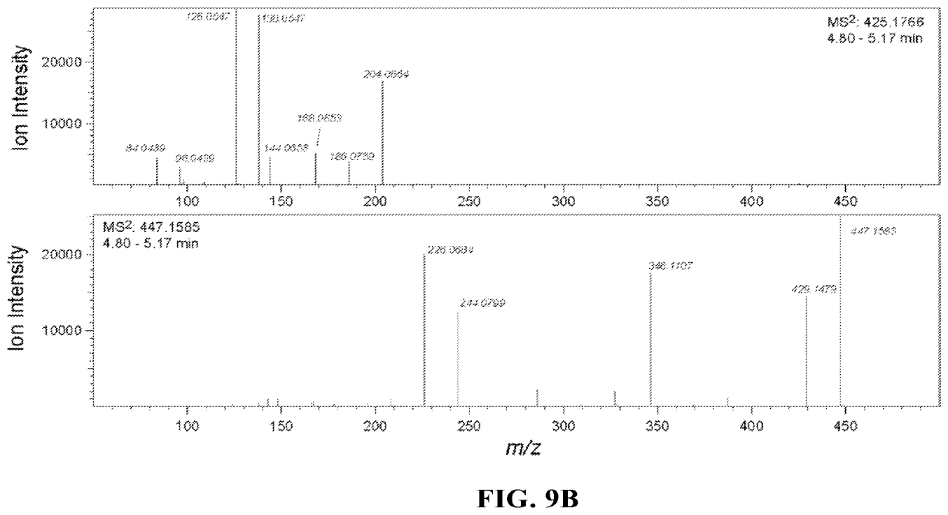

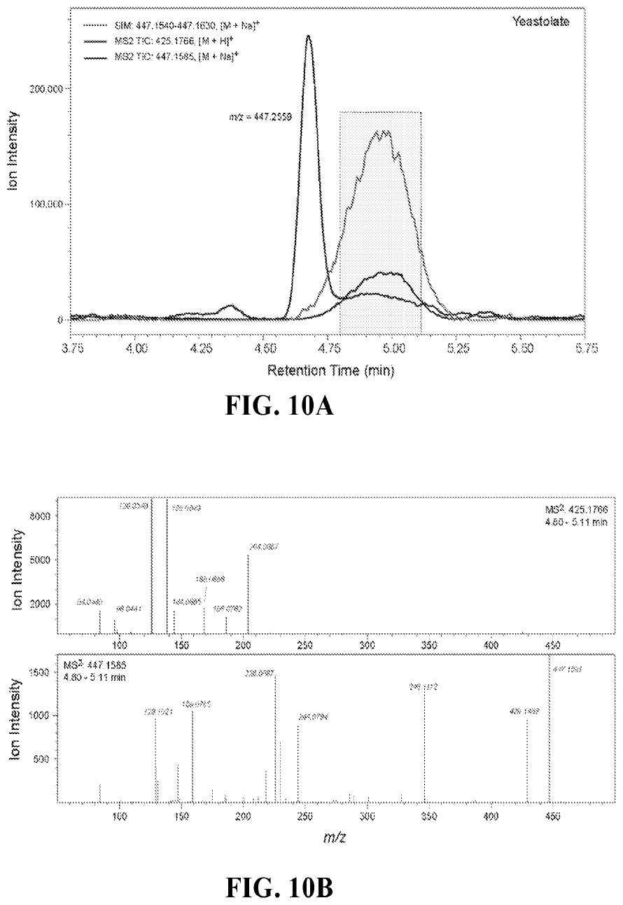

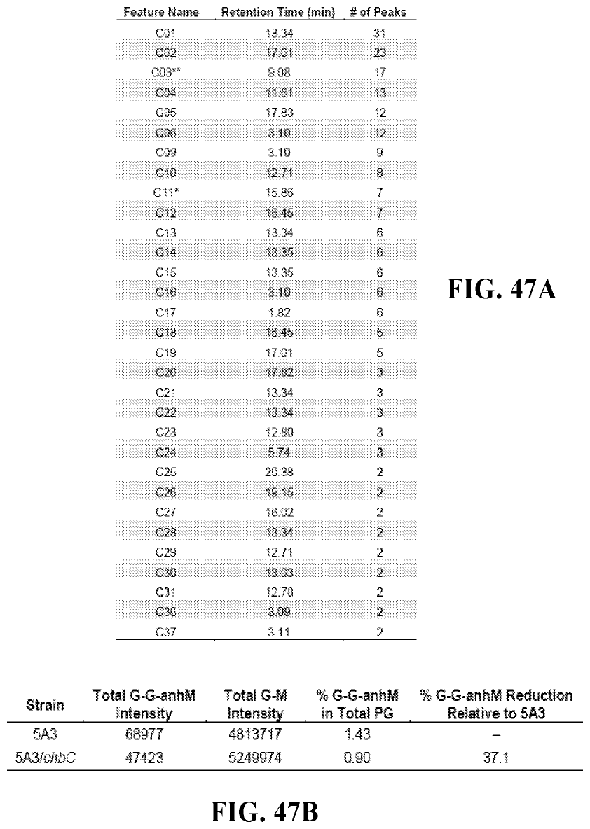

B. burgdorferi can survive in the I. scapularis tick midgut for months between feeding cycles, so nutrient-rich blood is not a consistent carbon source. A plausible carbon source other than a blood meal is chitin, the primary com-ponent of the tick peritrophic membrane²⁰. N,N′-Diacetylchitobiose (chitobiose) is the repeat unit of chitin, a disaccharide of GlcNAc with a β-(1-4) glycosidic linkage, which is also present in BSK-II culture medium, routinely used to grow Borrelia spp. (FIGS. 9A-9B and 10A-10B). The G-G-anhM sequence is essentially chitotriose with a 3-O-lactyl moiety. To assess the possibility that chitobiose is involved in B. burgdorferi peptidoglycan biosynthesis, we used a mutant bacterium (A3/chbC) that is incapable of importing GlcNAc-GlcNAc into the cytoplasm, as determined by isotopically labelled uptake experiments²¹. First, Applicant used WGS to confirm that the parental A3 strain (analysed earlier) and the A3/chbC mutant strain were clonal and, with the exception of the hypervariable vlsE locus²² and the targeted deletion of chbC gene, the strains were genetically identical (FIG. 46). Principal component analysis of the muropeptide profiles from three biological replicates—six different batches of culture—of the wild-type (WT) A3 and A3/chbC bacteria indicated homogeneity between replicates, but distinct features were apparent, suggesting that chitobiose transport impacts peptidoglycan composition (FIG. 3A). Comparative analysis of muropeptide identity and absolute abundance revealed that the parental strain contained more peptidoglycan per cell (FIG. 3B). Applicant's interpretation of these findings is that breakdown products of chitobiose are used to build the B. burgdorferi peptido-glycan cell wall^{13,15,21} and a lack of chitobiose reduces the amounts of peptidoglycan. Importantly, even after Applicant normalized for decrease in peptidoglycan, Applicant found that bacteria that were unable to import chitobiose from their environment had ˜37% less G-G-anhM (FIG. 47A-47B). These data suggest that one source of G-G-anhM is chitobiose, and Applicant would note that in a tick the only source of chitobiose would be the tick itself.

### Peptidoglycan Defects in the Absence of Chitobiose.

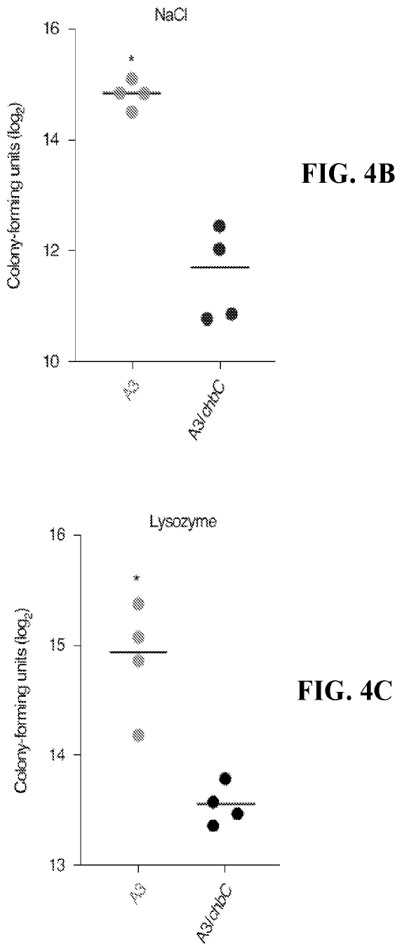

Bacteria rely on peptidoglycan as an osmoprotectant and a load-bearing structure. Applicant hypothesized that severe phenotypes would result from reduced peptidoglycan and/or G-G-anhM. Applicant used atomic force microscopy (AFM) to analyse purified peptidoglycan sacculi and found that A3/chbC peptidoglycan was jagged and frayed, com-pared with smooth, WT, peptidoglycan sacculi (FIG. 4A). The gross structural defects that we observed in purified peptidoglycan sacculi from A3/chbC led us to ascertain the phenotypes of live cells. Applicant exposed parental WT A3 and A3/chbC strains to either osmotic (NaCl; FIG. 4B) or peptidoglycan-specific (lysozyme; FIG. 4C) stressors for 24 h, diluted each into medium lacking stress and plated. The parental A3 control strain produced significantly more colonies, indicating that it was able to withstand osmotic and enzymatic degradation better than the mutant (FIG. 4B-4C).

Motility and Physical Properties of Peptidoglycan with Fewer GlcNAc-GlcNAc Disaccharides.

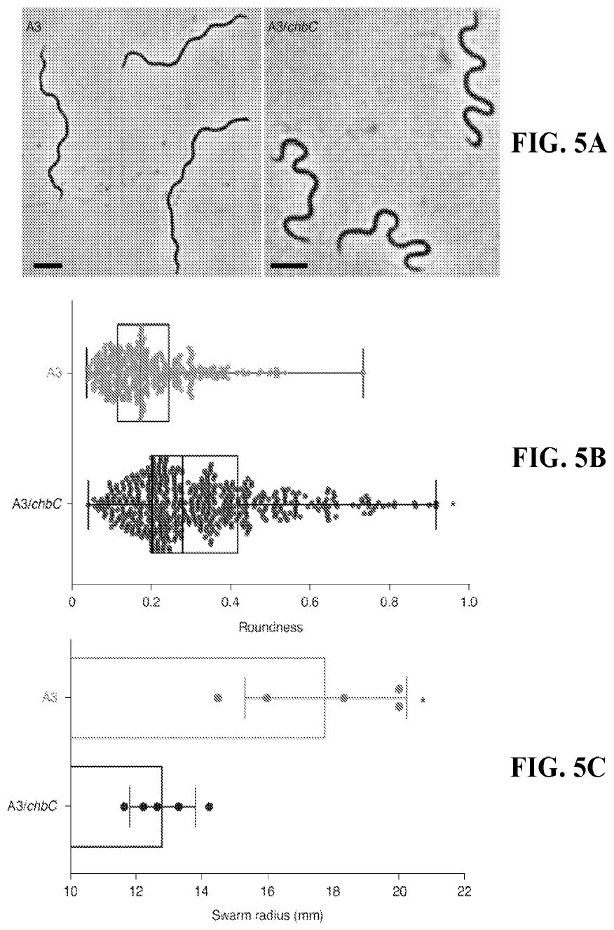

One distinguishing feature of Borrelia spp. is periplasmic flagella. An individual flagellum wraps around the cell cylinder and peptidoglycan layer to impart a ‘flat-wave’ morphology²³. Each flagellum is inserted into 7-11 motors²⁴, which are positioned adjacent to each cell pole. Motor rotation of the flagella produces huge torsional stress, which creates backward moving waves that propel the organism forwards. Theoretically, contorting the cell cylinder with torque of this magnitude would necessitate strong and flexible peptidoglycan to counteract the deforming forces produced by the flagella^{25,26}. Applicant speculated that defects in peptidoglycan caused by a reduction in G-G-anhM might alter the response to flagellar ribbon tension, thereby resulting in altered morphology. Phase-contrast micrographs of individual cells show a clear discrepancy in the pitch (or trough) of the wave between WT A3 and A3/chbC strains (FIG. 5A). Morphometric, single-cell analysis between each population was determined by measuring the Roundness²⁷ or the collective area required to enclose an object in an ellipse, corrected by aspect ratio^{28,29}. Roundness provides a normalized assessment of deviations from the typical flat-wave morphology by estimating collective differences in helical trough depth. Population-level analysis of individual cells confirmed that there was a significant amount of variability in the helical pitch of the chitobiose mutant strain (FIG. 5B). Morphological changes in helicity suggest an imbalance in the elastic force homoeostasis between the peptidoglycan and the motility machinery.

An imbalance in counteracting forces may impact spirochaete motility. Applicant evaluated this possibility by a swarm assay, in which two equidistant sites on a single semisoft agar plate were inoculated with each strain and, after 5 d of incubation, the radial distance was measured. Although A3/chbC retains the ability to move in this assay, the A3 WT strain translated significantly greater distances, confirming that cell wall:motility balance was disrupted (FIG. 5C).

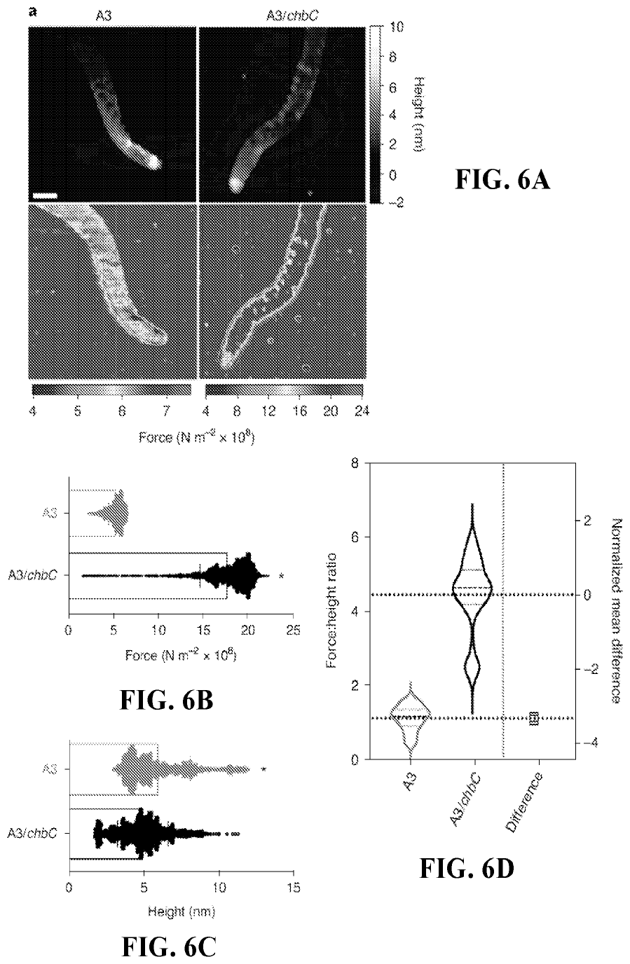

These data lend support to a model in which B. burgdorferi peptidoglycan homoeostasis is tuned to the torsional stress created by periplasmic flagella³⁰. These analyses provide evidence that the B. burgdorferi cell-wall composition is required to withstand the torsional forces produced by periplasmic endoflagella. The phenotypic differences (FIG. 4A-4C and 5A-5C) Applicant observed in bacteria unable to import chitobiose might result solely from reduced levels of cellular peptidoglycan (FIG. 3B), or it is possible that reduced levels of G-G-anhM could alter the biophysical properties of the B. burgdorferi peptidoglycan sacculus. To evaluate whether the incorporation of GlcNAc-GlcNAc into B. burgdorferi peptidoglycan increases the distance between muropeptides, adjacent to glycan termini (FIG. 2A-2D and FIG. 8A-8B), and renders peptidoglycan more flexible, Applicant carried out elasticity-based mechanical measurements using AFM on purified sacculi. Comparative analysis of peptidoglycan elasticity between individual sacculi appeared similar (FIG. 6A) but, to capture the full range of measurements, the A3/chbC sample required a colour map that extended >3× the maximum force of WT sacculi (FIG. 6A). Tandem height and force-map measurements revealed that peptidoglycan samples with reduced G-G-anhM were, on average, 3.3× stiffer than those with WT levels of G-G-anhM (FIG. 6B). We note, however, that topological height mapping also showed differences in peptidoglycan thickness between samples (FIG. 6C), potentially due to less total peptidoglycan in A3/chbC (FIG. 6B). To exclude the possibility that differences in thickness contribute to elastic modulus differences, we normalized readouts to peptidoglycan thickness, and performed a relative comparison on the same sacculi. Even after accounting for thickness differences, G-G-anhM content correlated with elasticity (FIG. 6D).

### Discussion

Peptidoglycan is important in bacterial physiology, morphology, cell biology, host interactions, and as a target for antibiotics_{1,31,32}. Peptidoglycan cell-wall chemistry is intimately linked to each process, but typically by way of the variability in muropeptide(s) and/or their linkages. Peptidoglycan glycan stoichiometry, on the other hand, was thought to be invariable. In the present study, we report that peptidoglycan from multiple strains and species of Borrelia terminates glycans with G-G-anhM (FIG. 2D).

Peptidoglycan determines the shape of most bacterial cells through its flexibility and structure^{33,34} However, in B. burgdorferi, the periplasmic flagellar ribbon is the main cell-shape determinant^{23,24}. Modelling has indicated that peptidoglycan resists both the natural curvature of the flagellar filaments and the stress created by locomotion^{25,35}. Applicant provides evidence for this model. Specifically, Applicant demonstrated that bacteria unable to import chitobiose have reduced amounts of peptidoglycan (FIG. 3A-3B) and altered peptidoglycan composition (FIG. 4A-4C), which results in abnormalities in cell morphology (FIG. 5A-4B).

Disrupting the balance between flagellar motion and peptidoglycan structure impairs motility (FIG. 5C). The elastic properties of peptidoglycan are a function of the degree and type of peptide cross-linking, in addition to the thickness and glycan orientation relative to the long axis^{33,36-38}. Applicant's AFM analyses suggest that a B. burgdorferi peptidoglycan structure might endow cells with elasticity by terminating glycans with G-G-anhM (FIGS. 1A-1C and 2A-2D), which enables larger distances between adjacent peptides and increases peptidoglycan flexibility (FIG. 6A-6D). The latter is remarkable given that, on average, B. burgdorferi glycan length is 30 disaccharides⁷ and, thus, G-G-anhM can constitute only ˜3.3% of all muropeptides (FIGS. 12-14). Although it is difficult to make direct comparisons of elastic modulus results due to the variability in probe standardization and data acquisition, our Young's modulus measurements of WT B. burgdorferi peptidoglycan (˜5.2×108 N m⁻²; FIG. 6B) are very much in line with those published for E. coli (˜3.5×108 N m⁻²)³⁶, which were obtained using similar sample processing and data acquisition conditions. The elastic properties of peptidoglycan isolated from bacteria unable to utilize chitobiose—leading to a reduction in G-G-anhM—were reduced, resulting in stiffer peptidoglycan (FIG. 6A-6D). Applicant's data support the hypothesis that the flexibility and molecular organization of the B. burgdorferi cell wall are fine-tuned to the shape-determining properties of periplasmic flagella to enable optimal motility³⁸.

The Lyme disease spirochaete lives in two distinct environments: vertebrates and ticks³⁹. The chbC transcript is expressed during all phases of growth⁴⁰, and is upregulated in the tick vector⁴¹ and under conditions similar to the tick midgut¹³, when spirochaete replication rate is slow⁴² and sugar metabolism is at a premium. The tick-associated, B. burgdorferi response regulator Rrp1 is involved in chbC upregulation in the vector, probably via RpoS15. Chitobiose is thought to be important both in cell-wall biosynthesis and as a carbon/nitrogen source in the nutrient-poor tick midgut, but through the utilization and isomerization of GlcNAc monomers^{13,15,23} not the direct use of the disaccharide chitobiose in peptidoglycan biosynthesis (FIG. 3A-3B). It is surprising that chitobiose transport is not required to successfully complete the tick-vertebrate enzootic life cycle of B. burgdorferi²¹. Chitobiose transport accounts for only ˜37% of peptidoglycan G-G-anhM (FIG. 47A-47B), which means that B. burgdorferi must possess additional, yet to be determined, means by which G-G-anhM is synthesized.

B. burgdorferi encounters transient changes in osmotic stress during migration from the tick midgut to the salivary glands during feeding and subsequently in a vertebrate host⁴³. Bacteria with reduced chbC synthesis cannot survive in medium with >500 mos-mol⁴³, which is in line with our findings (FIG. 4B). Curiously, early stages of B. burgdorferi migration in the tick are reported to coincide with changes in spirochaete morphology and mode of motility⁴⁴. It is possible that, similar to other pathogens that alter their peptide cross-linking to withstand changes in environmental and host-derived insults⁴⁵, B. burgdorferi alters the amount of G-G-anhM in its cell wall during different stages of the enzootic cycle.

Bacterial growth requires peptidoglycan turnover. Fragments are excised from the existing sacculus and replaced with large multimers, resulting in elongation. Instead of re-purposing released muropeptides, like many diderms B. burgdorferi sheds them into their environment⁷. The hallmark of muropeptide turnover is the release of anhMurNAc-containing peptidoglycan frag-ments⁴⁶. It is tempting to speculate that G-G-anhM may be key in peptidoglycan-associated Lyme disease pathologies⁷. Not only may G-G-anhM-containing muropeptides produce unusual innate immune-mediated responses, but also they may be responsible for creating specificity in certain surveillance system(s)⁴⁷. The unusual sugar organization may also be more resistant to degradation (FIG. 4C) by host-derived lysozyme and could be key in extending the half-life of B. burgdorferi peptidoglycan in the synovial fluid of patients with Lyme disease arthritis⁷.

The evolutionary landscape of arthropods, and their resident microbial symbionts, is beginning to come into focus. Co-evolutionary adaptive mechanisms have been fine-tuned for tens of millions of years⁴⁸. For instance, I. scapularis has co-opted a peptidoglycan hydrolase of bacterial origins to limit B. burgdorferi expansion⁴⁹, while protecting itself from pathogen acquisition. Microbial communities act in concert to alter tick midgut physiology, impacting the frequency and transmissibility of its residents^{50,51}. B. burgdorferi has foregone the need for seemingly essential vitamins like thiamine, which are probably not present in tick midguts⁵². Applicant's findings provide another example of how an endoparasitic bacterium has evolved to hijack arthropod components for use as a basic cell-wall building block.

### Methods

B. burgdorferi Strains, Genome Analysis, Growth Conditions and Analysis.

All B. burgdorferi strains used in the present study are transformable derivatives of the type strain10. B. burgdorferi B31-5A11, B31-5A3 and a non-infectious clone of B31-5A3 (ref 53) were provided by F. Gheradini (National Institutes of Health (NIH)), J. Coburn (Medical College of Wisconsin) and U. Pal (University of Maryland), respectively. The B31-5A3/chbC1 strain was provided by P. Rosa (Rocky Mountain Labs, NIH) and has been characterized elsewhere²¹. B. hermsii strain HS1 was purchased from American Type Culture Collection (ATCC).

All Borrelia strains were grown in Barbour-Stoenner-Kelly II (BSK-II) medium supplemented with 6% heat-inactivated rabbit serum (Gibco Laboratories), hereafter referred to as BSK-II complete culture medium⁵⁴. Applicant notes that BSK-II complete culture medium contains yeast autolysate, which is a source of chitobiose (FIG. 9A-9B and 10A-10B). Metabolic labelling studies simply replaced unlabelled GlcNAc (Sigma-Aldrich, 0.33 g l⁻¹) with [1-¹³C]GlcNAc or [1-¹³C]ManNAc (Omicron Biochemicals). GlcNAc-free BSK-II was supplemented with varying amounts of GalNAc or ManNAc (Sigma-Aldrich), as described in the text. Regardless of medium manipulations, all cultures were incubated at 37° C. with 5% CO₂. Bacteria were enumerated using Incyto C-Chip disposable haemocytometers (SKC Inc.). All measurements were performed in triplicate and the average was reported or used to normalize material for downstream analysis.

The entire genome of each B. burgdorferi strain was sequenced to confirm: (1) plasmid content; (2) clean deletion of A3/chbC; (3) A3/chbC free of polar mutations; and (4) clonality (FIGS. 45-46). Applicant notes that cells collected for DNA analysis were from a small fraction of a larger batch of culture that was used for peptidoglycan analysis and, thus, were the same passage. For instance, 450 ml of batch culture was split into 40 ml and 410 ml before harvesting cells by centrifugation. After washing each 3× with phosphate-buffered saline (PBS), genomic DNA was purified using quick-DNA miniprep plus kit (Zymo Research) following the manufacturer's recommended procedures for the 40-ml culture, whereas the rest was used to attain a highly pure preparation of peptidoglycan (below). Purified DNA was sequenced and assembled by the Microbial Genome Sequencing Center. Reads were analysed using breseq⁵⁵ (freely available online at http://barricklab.org/breseq) to align Illumina reads with reference genome. Applicant ran breseq separately for each of three strains to identify base-pair substitutions and plasmid profiles relative to the reference genome B. burgdorferi B31 clonal isolate 5A3 (RefSeq GCF_000008685.2). Outputs were analyzed manually and summarized in FIGS. 45-46.

For peptidoglycan purification, cells were harvested when cultures reached a density of ˜5×10⁷ cells ml⁻¹ by centrifugation at 3,500 g for 15 min at 4° C. The resulting pellet was gently washed 3× with PBS before being centrifuged at 3,000 g for 15 min at 4° C. Whole-cell lysate pellets were stored at −20° C. for later use. For direct comparative purposes (A3 versus A3/chbC), cells were enumerated and peptidoglycan was extracted (below) from equivalent cell counts (5×10⁷ cells ml⁻¹).

### Peptidoglycan Isolation: Intact Peptidoglycan Sacculi.

Peptidoglycan was isolated and purified from 0.25-2 l of mid-log phase cultures; volumes depended on application. Regardless of culture volume, all peptidoglycan was prepared following previously published procedures^{7,56}. The final pellet, containing intact peptidoglycan sacculi, was resuspended in 495 μl of ultra-pure H2O. Intact peptidoglycan sacculi were stored at 4° C. for AFM analysis or used to generate digested muropeptides as described in the following sections.

### Peptidoglycan Processing for Muropeptide Analysis.

Intact peptidoglycan sacculi, resuspended in NaHPO4/NaH2PO4 buffer (5 mM, pH 5.5) containing mutanolysin (7.8 μl, 4000 U ml⁻¹; Sigma-Aldrich), were digested overnight at 37° C. with shaking. The following morning, an additional 7.8 μl of mutanolysin was spiked in and allowed to incubate, shaking, for 5 h at 37° C. The mutanolysin digest was then heat inactivated at 100° C. for 10 min. After heat inactivation, the digest was cooled to room temperature and centrifuged at 22,000 g for 30 min. The supernatant (containing digested peptidoglycan muropeptides) was carefully moved to a preweighed microfuge tube without disturbing the pellet (undigested peptidoglycan). The supernatant, containing digested muropeptides, was dried and the final weight determined.

Purified, dried muropeptides were fully dissolved in 150 μl of saturated sodium borate buffer, pH 9.25. Sodium borohydride or borodeuteride (50 mg) was dissolved in 500 μl of LC-MS-grade H₂O, and an aliquot (50 μl) was added slowly to the muropeptide solution with mixing after the addition was complete. The reduction was quenched after 1 h by the addition of LC-MS-grade formic acid (˜10 μl) to a pH of ˜3 as evaluated by pH paper. Samples were then immediately snap-frozen and dried using a high vacuum line equipped with a liquid nitrogen solvent trap. Dried samples were stored desiccated until analysis, which involved reconstitution in H₂O:MeCN (200 μl, 9:1, v:v) containing 0.1% formic acid. Reconstituted samples were sonicated in a water bath for 10 min and centrifuged at 4° C. (13,000 g, 10 min). From the centrifuged sample, 180 μl was placed in a labelled LC-MS vial for analysis.

### LC-MS Analysis.

Analyses were performed on a Shimadzu LCMS9030 QToF instrument interfaced with a LC-40B X3 UPLC, a SIL-40C X3 autosampler (10° C.) and a CTO-40C column oven (40° C.). Gradient separations utilized a BEH C18 column (2.1 mm×50 mm, 1.7-μm particle size; Waters) with solvent A (0.1% formic acid in water) and solvent B (0.1% formic acid in MeOH) at a constant flow rate of 0.4 ml min-1. Initial solvent conditions were 99:1 (A:B) which was held constant for 3 min, followed by a shallow linear gradient to 8% B at 12 min, then to 20% B at 24 min and finally to 95% B at 25 min, which was held for 4 min. The gradient was converted to starting conditions with a 1-min gradient to 1% B (29 min), followed by a 5-min hold. Sample injection volumes ranged from 0.5 μl to 20 1 μl. The first 1.25 min of the separation was diverted to waste to avoid reduction reaction product contamination of the mass spectrometer interface.

The mass spectrometer was operated in positive ion mode using electrospray ionization and external calibration (NaI). Interface voltage was 4.0 kV at 300° C., with a desolvation temperature of 526° C. and a DL transfer line temperature of 250° C. Gas flows (1 min⁻¹) were 2, 10 and 10 for nebulizing, heating and drying gases, respectively. Muropeptide data were collected between 1.25 and 24 min using several different MS and MS/MS programmes. For statistical comparisons of strains, data were collected in MS mode only, from 400 m/z to 2,000 m/z at 0.1 Hz. Fragmentation data were collected in data-dependent mode (top three) at low Q1 resolution, with three MS/MS spectra, before placement on the exclusion list (15 s of exclusion time). The precursor window as set to 400-2,000 m/z with fragmentation data collected between 50 and 2,000 m/z, using a ramped collision energy (25±10 V). Total duty cycle was 0.4 s (0.1 s per event).

### Lc-MS Data Analytics.

Shimadzu.LCD files were converted to the .mzML file format using Shimadzu LabSolutions (v.5.99 SP2). The discovery of features and associated peak areas was performed using the xcms package (v.3.13) in the R programming environment (v.4.0.3)^{57,58}. The R package RamClustR (v.1.1)⁵⁹ was used to reduce spectral redundancy through the binning of the features into groups and this reduced dataset was used for further statistical analysis. Statistical analysis was performed using MetaboAnalyst 4.0 (ref. 60). Principal component analysis was performed on log(transformed) and pareto-scaled peak area values.

To determine the relative amount of G-G-anhM present in WT A3 and A3/chbC strains, Applicant prepared 6 independent, 450-ml culture volumes of BSK-II complete medium. Each strain was propagated in three independent cultures and peptidoglycan was purified from the same number of cells, in all six samples. Relative abundances of muropeptides were quantified from all three independent peptidoglycan samples of A3 and A3/chbC. RamClustR was once again used as above; however, from the resulting binned dataset, data were manually curated to ensure that all adducts and redundancies were successfully filtered out. For each muropeptide, from each replicate, relative abundance was calculated as the amount of muropeptide compared with the sum of all muropeptides present. These values were averaged between all three replicates of A3/chbC and compared with the averaged replicates of A3 for each muropeptide.

### Confirmation of Chitobiose in Autohydrolysed Yeast.

The confirmation is based on matching retention times and high-resolution mass spectrometric analysis of both parent and fragment ions. Both a chitobiose standard (Neogen) and autohydrolysed yeast (Yeastolate, Difco, BD & Co.) were separated by porous graphitic carbon (PGC) LC, essentially as described previously⁶¹. Separations were performed on a Hypercarb PGC column (100 mm×2.1 mm, 5-mm particle size; Thermo Fisher Scientific) using a binary gradient of water (solvent A) and acetonitrile (solvent B), both containing 10 mM ammonium hydroxide. The separation began at 95% solvent A (0-2 min), with a linear gradient to 75% A at 15 min and then to 5% A at 20 min. The system was held at 5% A for 4 min, with a 1-min linear ramp back to initial conditions, and held for 5 min. Total run time was 30 min at a flow rate of 0.4 ml min-1, with the column maintained at 50° C. The LC unit comprised two LC-40B X3 pumps, a SIL-40C X3 autosampler (10° C.) and a CTO-40C column oven (Shimadzu Scientific). The first 1.25 min of the separation was sent to waste, with data collection from 1.25 min to 24 min.

The mass spectrometer (LCMS9030; Shimadzu) was operated as described for the muropeptide work using three events. Event 1 was a scan from 200 m/z, to 1,500 m/z, followed by two sequential MS/MS scans using m/z values matching those of the [M+H]⁺ and [M+Na]⁺ forms of N,N′-diacetylchitobiose (425.1766 and 447.1585). Each event time was 0.1 s. Collision energy for the MS/MS scans was ramped ±17 V centered on 35 V.

### Monosaccharide Analysis.

Peptidoglycan glycosyl composition analysis was performed by the Complex Carbohydrate Research Center (Athens, GA). Peptidoglycan was purified, as described above, from two independent E. coli K-12 and B. burgdorferi 5A11 cultures. Each sample was spiked with 20 μg of myoinositol (internal standard) and hydrolyzed (200 μl 6 M HCl, 100° C., 16 h). After solvent removal under a stream of nitrogen, glycosyl composition analysis was performed by combined gas chromatography (GC)-MS of the alditol acetates as described previously62. The samples were hydrolyzed again in 2 M trifluoroacetic acid for 2 h in a sealed tube at 120° C., reduced with NaBD₄ and acetylated using acetic anhydride/pyridine. The resulting alditol acetates were analyzed by GC-MS analysis on an Agilent 7890A GC interfaced to a 5975C MSD, electron impact ionization mode. Separation was performed on a 30-m Equity 1 capillary column. Alongside the samples, standards of GlcNAc, GalNAc, ManNAc and MurNAc were also analyzed.

### NMR Spectroscopy.

Muropeptide samples and a chitotriose standard were reduced with NaBH₄ as described above, followed by removal of reaction byproducts using gravity-fed size exclusion chromatography (1 cm×20 cm column, 15 ml of Bio-Gel P-2 medium, fine-grade) using a 9:1 (v:v) mixture of water:95% ethanol (food grade/glass distilled) as the mobile phase. Muropeptide fractions (˜0.5 ml) were collected manually and combined after assessment by ultraviolet absorption (DeNovix DS-11 FX+) and LC-MS. Combined fractions were snap-frozen, dried and freeze-dried once with 100% D2O before NMR. Samples (unlabelled muropeptides, ¹³C-labelled and chitotriitol) were dissolved in 100% D2O, placed in a standard NMR tube (unlabelled and chitotriitol) or a Shigemi tube (¹³C-labelled) and analysed on a Bruker Biospin600 MHz instrument. Standard pulse sequences were used for ¹H, ¹³C, COSY, gH2BC, gHMBC and gHSOC. Data were processed using MestReNova (v.14.2, Mestrelab Research).

### Stress Tests and Plate Recovery Assay.

WT 5A3 and 5A3/chbC strains were cultured to a final density of 5×10⁷ cells ml-1 and back-diluted to a concentration of 10⁶ cells ml⁻¹ in 5 ml in BSK-II complete culture medium. NaCl (Affymetrix) and lysozyme (Sigma-Aldrich) were added to a final concentration of 0.1 M and 0.375 μg ml⁻¹, respectively—one treatment per tube, per strain—and incubated for 24 h at 37° C. and 5% CO₂. These conditions are identical to those used previously for a similar purpose8. Applicant notes that the addition of 0.1 M NaCl resulted in a final osmolality of 544 mosmol, as determined by Fiske Micro-Osmometer Model 210, following the manufacturer's recommended procedure.

Four batches of 100-ml BSK plating medium were prepared as previously described8 and added to four 100-ml volumes of pre-equilibrated, 5% low-melt agarose solution (1:1 ratio). The resulting solution was then allowed to re-equilibrate to 48° C. in the water bath (referred to as the plating medium hereafter). The plating medium was poured—25 ml per plate, 4 plates per batch of plating medium, for 16 plates—and allowed to solidify at room temperature for 2 h. The medium poured constitutes the bottom layer. The top layer consisted of an equal amount of culture medium, which was inoculated in a serial dilution of each strain, for each treatment. After 9 d, the colony-forming units were determined using a magnified Petri dish light box, fitted with a grid.

The serial dilution replicates for each strain and treatment were normalized by cell inoculum concentration, to the highest concentration, and reported as total colony-forming units observed. Differences in total colony-forming units were compared using a two-tailed, unpaired Student's t-test and graphed in GraphPad Prism 8.0.

### Swarm Motility Assay.

To evaluate the motility of 5A3 and 5A3/chbC strains, we prepared solid medium as described above, but with the following modifications: (1) the final concentration of low-melt agarose was adjusted to 0.5% (w:v) and (2) the entire volume-plating medium was added at once (that is, no layering) and allowed to solidify for 4 h at room temperature. After the plates solidified, we subsurface inoculated one side of each plate with 7.5 μl of the 10⁹ cells ml⁻¹ of 5A3, and the other with equal amounts of 5A3/chbC. This was repeated for a total of five plates.

After 5 d we measured the radius, in millimetres, of the disseminated colony in four different directions. These values were averaged to obtain a single, average radius value, which was recorded for both strains on each plate. Differences in the five average radius values for 5A3 and 5A3/chbC were compared using a two-tailed, unpaired Student's t-test and graphed in GraphPad Prism 8.0.

### Sample Preparation, Brightfield Microscopy and Image Analysis.

The morphological differences between 5A3 and 5A3/chbC were evaluated using phase-contrast microscopy on fixed cells. Briefly, both strains were cultured to a final density of 107 cells ml⁻¹ in BSK-II complete culture medium. Cells were fixed by adding 16% paraformaldehyde, from a fresh ampoule to a final concentration of 1.8% (v:v), as previously described⁶³. The mixture was incubated with gentle agitation for 10 min at room temperature, followed by 20 min on ice. Fixed cells were harvested by centrifugation at 3,500 g for 15 min at 4° C., and washed 3× with, and resuspended in, PBS.

Fixed cells were spotted on 2% agarose (in PBS) pads, as previously described⁵⁶. Phase-contrast micrographs were acquired on a Zeiss Axio Observer equipped with an oil-immersion phase-contrast objective Plan Apochromat 100×/1.45 numerical aperture (Nikon) using a Hamamatsu Orca-Flash 4.0 V3 Digital CMOS camera. Image acquisition occurred on the same day, using the same agarose pad, which was split in half. Cell preparation, image acquisition and analysis were repeated to ensure reproducibility. Results from independent experiments were almost identical and, thus, results from one experiment were reported.

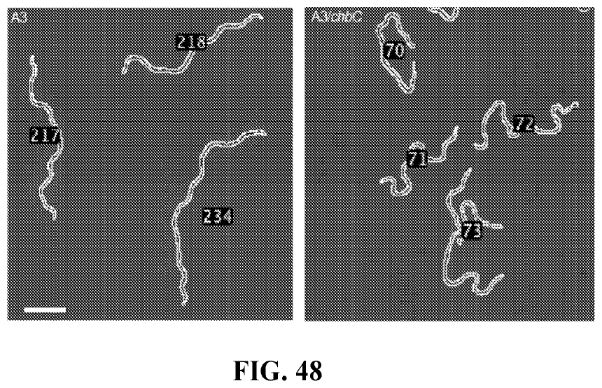

Applicant attempted to use the automated cell detection software Oufti64, as has been done in the past for B. burgdorferi phase-contrast micrographs56. However, the gross morphological changes (FIG. 5A-5C) in 5A3/chbC made cell detection challenging. We opted for an alternative approach whereby a threshold was applied to each phase-contrast micrograph, using Fiji. This resulted in clear cell outlines, with clean cell boundaries, for virtually all cells in a field of view (see FIG. 48, for example). After semi-automated cell detection, we used the macro function Roundness to calculate differences in cell shape. In the present study, the cell area is fitted to an ellipse, normalized by the aspect ratio of the object—an established method to evaluate the differences in the area that a cell occupies²⁷. Values were attained from ≥300 cells for each experiment and statistical significance was determined by an unpaired Student's t-test.

### AFM.

A suspension of purified peptidoglycan (above), isolated from 5A3 and 5A3/chbC, was created with ultra-pure water, diluted 1:5 (v:v), and 50 μl was deposited on to a freshly cleaved mica sheet (10 mm in diameter) attached to a metal AFM sample puck with epoxy. Samples were incubated for 5 min before being dried with nitrogen gas. All images were acquired using a Jupiter-XR AFM (Oxford Instruments Asylum Research) operating in amplitude-modulated-frequency-modulated (AM-FM) mode with an AC160TS-R3 (Olympus) cantilever. Cantilever oscillation was produced using photothermal excitation. The cantilevers first Eigen and second Eigen modes were tuned to free amplitudes of 2 and 0.025 V, respectively. The setpoints were established to achieve a phase angle <90° (repulsive regime) to permit stiffness image acquisition: typically, 1.5 and 0.018 V, respectively. Stiffness values were calculated using the Hertz contact model assuming that the radius of contact was 8 nm. Before image acquisition, the cantilever spring constant was calibrated using Asylum Research's GetReal Calibration Software API. Raw data files were processed and analyzed using Gwyddion. Height and stiffness measurements were compiled in Gwyddion and results graphed using GraphPad Prism 8.0.

### Data Availability

All data collected from our studies can be found in this Example and DeHart et al., Nature Microbiol. 6:1583-1592 (2021). The raw WGS data can be found here: strain B31-5A11, accession no. SAMN21566060 (https://www.ncbi.nlm.nih. gov/biosample/SAMN21566060; strain B31-5A3, accession no. SAMN21566061 (https://www.ncbi.nlm.nih.gov/biosample/21566061); strain B31-5A3n.i, accession no. SAMN21566062 (https://www.ncbi.nlm.nih.gov/biosample/21566062); strain B31-5A3/chbC, accession no. SAMN21566063 (https://www.ncbi.nlm.nih.gov/biosample/21566063).

### References for Example 1

1. Vollmer, W., Blanot, D. & de Pedro, M. A. Peptidoglycan structure and architecture. FEMS Microbiol. Rev. 32, 149-167 (2008).
2. Kugeler, K. J., Schwartz, A. M., Delorey, M. J., Mead, P. S. & Hinckley, A. F. Estimating the frequency of Lyme disease diagnoses, United States,

2010-2018. Emerg. Infect. Dis. 27, 616-619 (2021).
3. Branda, J. A. et al. Advances in serodiagnostic testing for Lyme disease are at hand. Clin. Infect. Dis. 66, 1133-1139 (2018).
4. Steere, A. C. Treatment of Lyme arthritis. J. Rheumatol. 46, 871-873 (2019).
5. Motaleb, M. A., Liu, J. & Wooten, R. M. Spirochetal motility and chemotaxis in the natural enzootic cycle and development of Lyme disease. Curr. Opin. Microbiol. 28, 106-113 (2015).
6. Charon, N. W. et al. The unique paradigm of spirochete motility and chemotaxis. Annu. Rev. Microbiol. 66, 349-370 (2012).
7. Jutras, B. L. et al. Borrelia burgdorferi peptidoglycan is a persistent antigen in patients with Lyme arthritis. Proc. Natl Acad. Sci. USA 116, 13498-13507 (2019).
8. Davis, M. M. et al. The peptidoglycan-associated protein NapA plays an important role in the envelope integrity and in the pathogenesis of the Lyme disease spirochete. PLoS Pathog. 17, e1009546 (2021).
9. Beck, G., Benach, J. L. & Habicht, G. S. Isolation, preliminary chemical characterization, and biological acitity of Borrelia burgdorferi peptidoglycan. Biochem. Biophys. Res. Commun. 167, 89-95 (1990).
10. Fraser, C. M. et al. Genomic sequence of a Lyme disease spirochaete, Borrelia burgdorferi. Nature 390, 580-586 (1997).
11. Barbour, A. G. Isolation and cultivation of Lyme disease spirochetes. Yale J. Biol. Med. 57, 521-525 (1984).
12. Schneider, E. M. & Rhodes, R. G. N-Acetylmannosamine (ManNAc) supports the growth of Borrelia burgdorferi in the absence of N-acetylglucosamine (GlcNAc). FEMS Microbiol. Lett. 365, (2018).
13. Tilly, K. et al. Genetics and regulation of chitobiose utilization in Borrelia burgdorferi. J. Bacteriol. 183, 5544-5553 (2001).
14. von Lackum, K. & Stevenson, B. Carbohydrate utilization by the Lyme borreliosis spirochete, Borrelia burgdorferi. FEMS Microbiol. Lett. 243, 173-179 (2005).
15. Sze, C. W. et al. Study of the response regulator Rrpi reveals its regulatory role in chitobiose utilization and virulence of Borrelia burgdorferi. Infect. Immun. 81, 1775-1787 (2013).
16. Elias, A. F. et al. Clonal polymorphism of Borrelia burgdorferi strain B31 MI: implications for mutagenesis in an infectious strain background. Infect. Immun. 70, 2139-2150 (2002).
17. Jewett, M. W. et al. The critical role of the linear plasmid lp36 in the infectious cycle of Borrelia burgdorferi. Mol. Microbiol. 64, 1358-1374 (2007).
18. Kawabata, H., Norris, S. J. & Watanabe, H. BBE02 disruption mutants of Borrelia burgdorferi B31 have a highly transformable, infectious phenotype. Infect. Immun. 72, 7147-7154 (2004).
19. Barbour, A. G. & Gupta, R. S. The family Borreliaceae (Spirochaetales), a diverse group in two genera of tick-borne spirochetes of mammals, birds, and reptiles. J. Med. Entomol. 58, 1513-1524 (2021).
20. Zhu, Z., Gern, L. & Aeschlimann, A. The peritrophic membrane of Ixodes ricinus. Parisitol. Res. 77, 635-641 (1991).
21. Tilly, K. G. D., Bueschel, D. M., Krum, J. G. & Rosa, P. Infectious cycle analysis of a Borrelia burgdorferi mutant defective in transport of chitobiose, a tick cuticle component. Vector Borne Zoonotic Dis. 4, 159-168 (2004).
22. Coutte, L., Botkin, D. J., Gao, L. & Norris, S. J. Detailed analysis of sequence changes occurring during vlsE antigenic variation in the mouse model of Borrelia burgdorferi infection. PLoS Pathog. 5, e1000293 (2009).
23. Motaleb, M. A. et al. Borrelia burgdorferi periplasmic flagella have both skeletal and motility functions. Proc. Natl Acad. Sci. USA 97, 10899-10904 (2000).
24. Charon, N. W. et al. The flat-ribbon configuration of the periplasmic flagella of Borrelia burgdorferi and its relationship to motility and morphology. J. Bacteriol. 191, 600-607 (2009).
25. Dombrowski, C. et al. The elastic basis for the shape of Borrelia burgdorferi. Biophys. J. 96, 4409-4417 (2009).
26. Goldstein, S. F., Charon, N. W. & Kreiling, J. A. Borrelia burgdorferi swims with a planarwaveform similar to that of eukaryotic flagella. Proc. Natl Acad. Sci. USA 91, 3433-3437 (1994).
27. Milner, D. S. et al. DivIVA controls progeny morphology and diverse ParA proteins regulate cell division or gliding motility in Bdellovibrio bacteriovorus. Front. Microbiol. 11, 542 (2020).
28. Takashimizu, Y. & Iiyoshi, M. New parameter of roundness R: circularity corrected by aspect ratio. Prog. Earth Planet. Sci. 3, 2 (2016).
29. Hart, M. et al. Shaping the cell and the future: recent advancements in biophysical aspects relevant to regenerative medicine. J. Funct. Morphol. Kinesiol. 3, 2 (2018).
30. Charon, N. W. & Goldstein, S. F. Genetics of motility and chemotaxis of a fascinating group of bacteria: the spirochetes. Annu. Rev. Genet. 36, 47-73 (2002).
31. Irazoki, O., Hernandez, S. B. & Cava, F. Peptidoglycanmuropeptides: release, perception, and functions as signaling molecules. Front. Microbiol. 10, 500 (2019).
32. Wolf, A. J. & Underhill, D. M. Peptidoglycan recognition by the innate immune system. Nat. Rev. Immunol. 18, 243-254 (2018).
33. Huang, K. C., Mukhopadhyay, R., Wen, B., Gitai, Z. & Wingreen, N. S. Cell shape and cell-wall organization in Gram-negative bacteria. Proc. Natl Acad. Sci. USA 105, 19282-19287 (2008).
34. Cabeen, M. T. & Jacobs-Wagner, C. Bacterial cell shape. Nat. Rev. Microbiol. 3, 601-610 (2005).
35. Goldstein, S. F., Buttle, K. F. & Charon, N. F. Structural analysis of the Leptospiraceae and Borrelia burgdorferi by high-voltage electron microscopy. J. Bacteriol. 178, 6539-6545 (1996).
36. Yao, X., Jericho, M., Pink, D. & Beveridge, T. Thickness and elasticity of Gram-negative murein sacculi measured by atomic force microscopy. J. Bacteriol. 181, 6865-6875 (1999).
37. Meroueh, S. O. et al. Three-dimensional structure of the bacterial cell wall peptidoglycan. Proc. Natl Acad. Sci. USA. 103, 4404-4409 (2006).
38. Harman, M. W. et al. Vancomycin reduces cell wall stiffness and slows swim speed of the Lyme disease bacterium. Biophys. J. 112, 746-754 (2017).
39. Radolf, J. D., Caimano, M. J., Stevenson, B. & Hu, L. T. Of ticks, mice and men: understanding the dual-host lifestyle of Lyme disease spirochaetes. Nat. Rev. Microbiol. 10, 87-99 (2012).
40. Arnold, W. K. et al. RNA-Seq of Borrelia burgdorferi in multiple phases of growth reveals insights into the dynamics of gene expression, transcriptome architecture, and noncoding RNAs. PLoS ONE 11, e0164165 (2016).
41. Pappas, C. J. et al. Borrelia burgdorferi requires glycerol for maximum fitness during the tick phase of the enzootic cycle. PLoS Pathog. 7, e1002102 (2011).
42. Jutras, B. L., Chenail, A. M. & Stevenson, B. Changes in bacterial growth rate govern expression of the Borrelia burgdorferi OspC and Erp [0343]infection-associated surface proteins. J. Bacteriol. 195, 757-764 (2013).
43. Bontemps-Gallo, S., Lawrence, K. & Gherardini, F. C. Two different virulence-related regulatory pathways in Borrelia burgdorferi are directly affected by osmotic fluxes in the blood meal of feeding Ixodes ticks. PLoS Pathog. 12, e1005791 (2016).
44. Dunham-Ems, S. M. et al. Live imaging reveals a biphasic mode of dissemination of Borrelia burgdorferi within ticks. J. Clin. Invest. 119, 3652-3665 (2009).
45. Yadav, A. K., Espaillat, A. & Cava, F. Bacterial strategies to preserve cell wall integrity against environmental threats. Front. Microbiol. 9, 2064 (2018).
46. Schaub, R. E. & Dillard, J. P. The pathogenic Neisseria use a streamlined set of peptidoglycan degradationproteins for peptidoglycan remodeling, recycling, and toxic fragment release. Front. Microbiol. 10, 73 (2019).
47. Gupta, A. et al. A human secretome library screen reveals a role for peptidoglycan recognition protein 1 in Lyme borreliosis. PLoS Pathog. 16, e1009030 (2020).
48. Stewart, P. E. & Bloom, M. E. Sharing the ride: Ixodes scapularis symbionts and their Interactions. Front. Cell. Infect. Microbiol. 10, 142 (2020).
49. Chou, S. et al. Transferred interbacterial antagonism genes augment eukaryotic innate immune function. Nature 518, 98-101 (2015).
50. Narasimhan, S. et al. Gut microbiota of the tick vector Ixodes scapularis modulate colonization of the Lyme disease spirochete. Cell Host Microbe 15, 58-71 (2014).
51. Abraham, N. M. et al. Pathogen-mediated manipulation of arthropod microbiota to promote infection. Proc. Natl Acad. Sci. USA 114, E781-E790 (2017).
52. Zhang, K. et al. Lyme disease spirochaete Borrelia burgdorferi does not require thiamin. Nat. Microbiol. 2, 16213 (2016).
53. Purser, J. E. & Norris, S. J. Correlation between plasmid content and infectivity in Borrelia burgdorferi. Proc. Natl Acad. Sci. USA 97, 13865-13870 (2000).
54. Zückert, W. R. Laboratory maintenance of Borrelia. Curr. Protoc. Microbiol. 12, C.1 (2007).
55. Deatherage, D. E. & Barrick, J. E. Identification of mutations in laboratory-evolved microbes from next-generation sequencing data using breseq. Methods Mol. Biol. 1151, 165-188 (2014).
56. Jutras, B. L. et al. Lyme disease and relapsing fever Borrelia elongate through zones of peptidoglycan synthesis that mark division sites of daughter cells. Proc. Natl Acad. Sci. USA 113, 9162-9170 (2016).
57. Tautenhahn, R., Bottcher, C. & Neumann, S. Highly sensitive feature detection for high resolution LC/MS. BMC Bioinforma. 9, 504 (2008).
58. R Core Team. R: A Language and Environment for Statistical Computing (R Foundation for Statistical Computing, 2009).
59. RAMClustR: Mass spectrometry metabolomics feature clustering and interpretation. R Package version 1.1.0 (2019).
60. Chong, J. et al. MetaboAnalyst 4.0: towards more transparent and integrative metabolomics analysis. Nucleic Acids Res. 46, W486-W494 (2018).
61. Fan, J. Q., Kondo, A., Kato, I. & Lee, Y. C. High-performance liquid chromatography of glycopeptides and oligosaccharides on graphitized carbon columns. Anal. Biochem. 219, 224-229 (1994).
62. Pena, M. J., Tuomivaara, S. T., Urbanowicz, B. R., O'Neill, M. A. & York, W. S. Methods for structural characterization of the products of cellulose- and xyloglucan-hydrolyzing enzymes. Methods Enzymol. 510, 121-139 (2012).
63. Brock, A. M. & Jutras, B. L. A simple method to detect Borrelia burgdorferi sensu lato proteins in different sub-cellular compartments by immunofluorescence. Ticks Tick. Borne Dis. 12, 101808 (2021).
64. Paintdakhi, A. et al. Oufti: an integrated software package for high-accuracy, high-throughput quantitative microscopy analysis. Mol. Microbiol. 99, 767-777 (2016).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A method of detecting a Borrelia burgdorferi (B. burgdorferi) organism or an infection thereof, the method comprising: detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide.
2. The method of aspect 1, wherein detecting comprises mass spectrometry, chromatography, a polymerase chain reaction (PCR)-based assay, an immunoassay, immunoseparation, electrophoresis, a periodate reaction, size-based separation, a mass separation technique, a charge separation technique, resonance spectroscopy, Raman spectroscopy, or any combination thereof.
3. The method of any one of aspects 1-2, wherein detecting comprises a PCR-immunoassay.
4. The method of any one of aspects 1-3, wherein detecting comprises contacting the sample with an antibody or fragment thereof capable of specifically binding the B. burgdorferi-specific peptidoglycan or fragment thereof.
5. The method of aspect 4, wherein the antibody or fragment thereof comprises a polypeptide having a sequence according to SEQ ID NO: 1, SEQ ID NO: 2, or both.
6. The method of any one of aspects 1-5, wherein the sample is a biological fluid sample.
7. The method of any one of aspects 1-6, wherein the biological fluid sample is whole blood, plasma, serum, saliva, synovial fluid, cerebrospinal fluid, urine, lymph, sweat, stool, mucus, tears, or any combination thereof.
8. The method of any one of aspects 1-7, wherein the sample is from a subject having, has had, or is suspected of having Lyme's disease and/or B. burgdorferi infection.
9. The method of any one of aspects 1-8, wherein the method is effective in detecting B. burgdorferi during one or more stages of B. burgdorferi infection.
10. The method of aspect 9, wherein the method is effect in detecting B. burgdorferi during any stage of B. burgdorferi infection.
11. The method of any one of aspects 1-10, further comprising diagnosing, monitoring, staging, and/or prognosing a B. burgdorferi infection and/or Lyme's disease or a symptom thereof in a subject from which the sample was obtained.
12. The method of any one of aspects 1-11, wherein the method does not detect other Borrelia species, other spirochetes, and/or other bacteria, and/or other microorganisms.
13. The method of any one of aspects 1-12, further comprising treating a B. burgdorferi infection, Lyme's disease, or both or a symptom thereof in a subject from which the sample was obtained by administering to the subject an anti-infective agent, an anti-inflammatory agent, an analgesic, an antibody or fragment thereof, or any combination thereof.
14. The method of aspect 13, wherein treating comprises administering an antibody or fragment thereof capable of specifically binding the B. burgdorferi-specific peptidoglycan or fragment thereof or a pharmaceutical formulation thereof to the subject.
15. The method of aspect 14, wherein the antibody or fragment thereof comprises a polypeptide having a sequence according to SEQ ID NO: 1, SEQ ID NO: 2, or both.
16. The method of any one of aspects 13-15, wherein the anti-infective agent comprises doxycycline, amoxicillin, cefuroxime, cefotaxime, azlocillin, penicillin, erythromycin, ceftriaxone, or any combination thereof.
17. The method of any one of aspects 1-16, further comprising staging Lyme's disease, infection with a B. burgdorferi organism, or both, or a symptom thereof.
18. A method of treating, diagnosing, prognosing, and/or staging Lyme's disease and/or infection with a Borrelia burgdorferi (B. burgdorferi) organism, and/or a symptom thereof, the method comprising: detecting, in a sample obtained from a subject that has had, has, or is suspected of having Lyme's disease and/or infection with B. burgdorferi, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; and administering an anti-infective agent, an anti-inflammatory agent, an analgesic, an antibody or fragment thereof, or any combination thereof.
19. The method of aspect 18, where the antibody or fragment thereof comprises one or more polypeptides each independently having a sequence according to SEQ ID NO: 1 or SEQ ID NO: 2.
20. The method of any one of aspects 18-19, wherein the anti-infective agent comprises of doxycycline, amoxicillin, cefuroxime, cefotaxime, azlocillin, penicillin, erythromycin, ceftriaxone, or any combination thereof.
21. The method of any one of aspects 18-20, wherein administering is oral, intermuscular, intravenous, intracerebroventricular, lumbar puncture, intra-articular, intraarterial, or intraperitoneal.
22. A kit comprising (a) one or more reagents capable of or adapted for detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; (b) one or more compositions or pharmaceutical formulations capable of treating a B. burgdorferi infection or a symptom thereof, wherein the one or more compositions or pharmaceutical formulations comprise an anti-infective agent, an anti-inflammatory agent, an analgesic, an antibody or fragment thereof, or any combination thereof, or (c) both (a) and (b).
23. The kit of aspect 22, wherein the antibody or fragment thereof comprises one or more polypeptides each independently having a sequence according to SEQ ID NO: 1 or SEQ ID NO: 2.
24. The kit of any one of aspects 22-23, wherein the one or more reagents capable of or adapted for detecting comprises an antibody or fragment thereof, wherein the antibody or fragment thereof comprises one or more polypeptides each independently having a sequence according to SEQ ID NO: 1 or SEQ ID NO: 2.
25. The kit of any one of aspects 22-24, wherein the anti-infective agent comprises doxycycline, amoxicillin, cefuroxime, cefotaxime, azlocillin, penicillin, erythromycin, ceftriaxone, or any combination thereof.
26. An antibody or fragment thereof comprising one or more polypeptides each independently having a sequence according to SEQ ID NO: 1 or SEQ ID NO: 2.
27. The antibody of aspect 26, wherein the antibody is capable of specifically binding a B. burgdorferi-specific peptidoglycan or fragment thereof.
28. The antibody of any one of aspects 26-27, wherein the antibody is capable of specifically binding B. burgdorferi-specific peptidoglycan or fragment comprising a GlcNAc-GlcNAc-MurNAc (GGM) trisaccharide.
29. A pharmaceutical formulation comprising an antibody or fragment thereof of any one of aspects 26-28; and a pharmaceutically acceptable carrier.
30. A method of treating a B. Burgdorefi infection or a symptom thereof in a subject in need thereof, the method comprising administering the antibody of any one of aspects 26-28 or a pharmaceutical formulation thereof to the subject in need thereof.

What is claimed is:

1. A method of detecting a Borrelia burgdorferi (B. burgdorferi) organism or an infection thereof, the method comprising:
detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide.

2. The method of claim 1, wherein detecting comprises mass spectrometry, chromatography, a polymerase chain reaction (PCR)-based assay, an immunoassay, immunoseparation, electrophoresis, a periodate reaction, size-based separation, a mass separation technique, a charge separation technique, resonance spectroscopy, Raman spectroscopy, or any combination thereof.

3. The method of claim 1, wherein detecting comprises a PCR-immunoassay.

4. The method of claim 1, wherein detecting comprises contacting the sample with an antibody or fragment thereof capable of specifically binding the B. burgdorferi-specific peptidoglycan or fragment thereof.

5. The method of claim 4, wherein the antibody or fragment thereof comprises a polypeptide having a sequence according to SEQ ID NO: 1, SEQ ID NO: 2, or both.

6. The method of claim 1, wherein the sample is a biological fluid sample.

7. The method of claim 6, wherein the biological fluid sample is whole blood, plasma, serum, saliva, synovial fluid, cerebrospinal fluid, urine, lymph, sweat, stool, mucus, tears, or any combination thereof.

8. The method of claim 1, wherein the sample is from a subject having, has had, or is suspected of having Lyme's disease and/or B. burgdorferi infection.

9. The method of claim 1, wherein the method is effective in detecting B. burgdorferi during one or more stages of B. burgdorferi infection.

10. The method of claim 9, wherein the method is effect in detecting B. burgdorferi during any stage of B. burgdorferi infection.

11. The method of claim 1, further comprising diagnosing, monitoring, staging, and/or prognosing a B. burgdorferi infection and/or Lyme's disease or a symptom thereof in a subject from which the sample was obtained.

12. The method of claim 1, wherein the method does not detect other Borrelia species, other spirochetes, and/or other bacteria, and/or other microorganisms.

13. The method of claim 1, further comprising treating a B. burgdorferi infection, Lyme's disease, or both or a symptom thereof in a subject from which the sample was obtained by administering to the subject an anti-infective agent, an anti-inflammatory agent, an analgesic, an antibody or fragment thereof, or any combination thereof.

14. The method of claim 13, wherein treating comprises administering an antibody or fragment thereof capable of specifically binding the B. burgdorferi-specific peptidoglycan or fragment thereof or a pharmaceutical formulation thereof to the subject.

15. The method of claim 14, wherein the antibody or fragment thereof comprises a polypeptide having a sequence according to SEQ ID NO: 1, SEQ ID NO: 2, or both.

16. The method of claim 13, wherein the anti-infective agent comprises doxycycline, amoxicillin, cefuroxime, cefotaxime, azlocillin, penicillin, erythromycin, ceftriaxone, or any combination thereof.

17. The method of claim 1, further comprising staging Lyme's disease, infection with a B. burgdorferi organism, or both, or a symptom thereof.

18. A method of treating, diagnosing, prognosing, and/or staging Lyme's disease and/or infection with a Borrelia burgdorferi (B. burgdorferi) organism, and/or a symptom thereof, the method comprising:
detecting, in a sample obtained from a subject that has had, has, or is suspected of having Lyme's disease and/or infection with B. burgdorferi, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide; and
administering an anti-infective agent, an anti-inflammatory agent, an analgesic, an antibody or fragment thereof, or any combination thereof or a pharmaceutical formulation thereof.

19. The method of claim 18, where the antibody or fragment thereof comprises one or more polypeptides each independently having a sequence according to SEQ ID NO: 1 or SEQ ID NO: 2.

20. The method of claim 18, wherein the anti-infective agent comprises doxycycline, amoxicillin, cefuroxime, cefotaxime, azlocillin, penicillin, erythromycin, ceftriaxone, or any combination thereof.

21. The method of claim 18, wherein administering is oral, intermuscular, intravenous, intracerebroventricular, lumbar puncture, intra-articular, intraarterial, or intraperitoneal.

22. A kit comprising:
(a) one or more reagents capable of or adapted for detecting, in a sample, a B. burgdorferi-specific peptidoglycan or fragment thereof, wherein the B. burgdorferi-specific peptidoglycan or fragment thereof comprises a GlcNAc-GlcNAc-MurNAc (GGM) trisaccharide;
(b) one or more compositions or pharmaceutical formulations capable of treating a B. burgdorferi infection or a symptom thereof, wherein the one or more compositions or pharmaceutical formulations comprise an anti-infective agent, an anti-inflammatory agent, an analgesic, an antibody or fragment thereof, or any combination thereof, or
(c) both (a) and (b).

23. The kit of claim 22, wherein the antibody or fragment thereof comprises one or more polypeptides each independently having a sequence according to SEQ ID NO: 1 or SEQ ID NO: 2.

24. The kit of claim 22, wherein the one or more reagents capable of or adapted for detecting comprises an antibody or fragment thereof, wherein the antibody or fragment thereof comprises one or more polypeptides each independently having a sequence according to SEQ ID NO: 1 or SEQ ID NO: 2.

25. The kit of claim 22, wherein the anti-infective agent comprises doxycycline, amoxicillin, cefuroxime, cefotaxime, azlocillin, penicillin, erythromycin, ceftriaxone, or any combination thereof.

26. An antibody or fragment thereof comprising:
one or more polypeptides each independently having a sequence according to SEQ ID NO: 1 or SEQ ID NO: 2.

27. The antibody of claim 26, wherein the antibody is capable of specifically binding a B. burgdorferi-specific peptidoglycan or fragment thereof.

28. The antibody of claim 26, wherein the antibody is capable of specifically binding B. burgdorferi-specific peptidoglycan or fragment comprising a GlcNAc-GlcNAc-MurNAc (GGM) trisaccharide.

29. A pharmaceutical formulation comprising:
an antibody or fragment thereof of claim 26; and
a pharmaceutically acceptable carrier.

30. A method of treating a B. burgdorferi infection or a symptom thereof in a subject in need thereof, the method comprising:
administering the antibody of claim 26 or a pharmaceutical formulation thereof to the subject in need thereof.


SEQUENCE LISTING (5 sequences)

SEQ ID NO: 1 - protein, 454 aa, synthetic construct
  source 1..454: note = Clone 1A4-H (G-G-anhM mAb) Heavy chain
METGLRWLLLVAVLKGVQCQSLEESGGRLVTPGTPLTLTCTVSGFSLSSYAVIWVRQAPGEGLEYIGIIFGGGKTYYASWAKGRFTISKTSTTVDLRITSPTTEDTATYFCVRDDWSRYLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 2 - protein, 239 aa, synthetic construct
  source 1..239: note = Clone 1A4-H (G-G-anhM mAb) Light chain
MDTRAPTQLLGLLLLWLPGATFAQVLTQTASPVSAAVGSTVTINCQSSESVYSNNYLSWFHQKPGQPPKQLIYSASTLASGVPSRFKGSGSGTQFTLTISDVQCDDAATYYCLGNYGCSSADCRAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 3 - protein, 10 aa, synthetic construct
  source 1..10: note = Nuclear export signal
  VARIANT 2..4: note = X is any amino acid
  VARIANT 6..7: note = X is any amino acid
  VARIANT 9: note = X is any amino acid
LXXXLXXLXL

SEQ ID NO: 4 - protein, 15 aa, synthetic construct
  source 1..15: note = Gly Ser linker
GGGGGGGGGGGGGGG

SEQ ID NO: 5 - protein, 15 aa, synthetic construct
  source 1..15: note = Gly Ser linker
GGGGSGGGGSGGGGS